(19) United States
(12) Patent Application Publication
(10) Pub. No.: US 2025/0034590 A1
(43) Pub. Date: Jan. 30, 2025

(54) METHODS AND SYSTEMS FOR TRANSFECTING HOST CELLS

(71) Applicant: Biogen MA Inc., Cambridge, MA (US)

(72) Inventors: Jenny Wang-Yuanzhen Shupe, Littleton, MA (US); Alexander Vaca, Cambridge, MA (US)

(21) Appl. No.: 18/717,244

(22) PCT Filed: Dec. 6, 2022
(86) PCT No.: PCT/US22/52000

§ 371 Date: Jun. 6, 2024

Related U.S. Application Data
(60) Provisional application No. 63/286,885, filed on Dec. 7, 2021.

(51) Int. Cl. C12N 15/86

(52) U.S. Cl. CPC C12N 15/86; C12N 2750/14143; C12N 2750/14152

(57) ABSTRACT

The present disclosure pertains to methods for transducing host cells with nucleic acids using an in-line complexer and systems comprising an in-line complexer for transducing host cells. The disclosed methods and systems can be used to produce recombinant adeno-associated virus (rAAV) particles. Also disclosed herein are compositions comprising rAAV particles obtained from the disclosed methods and systems, and uses of the same.

### CROSS-REFERENCE TO RELATED APPLICATIONS

This application claims priority to U.S. Provisional Patent Application 63/286,885 filed on Dec. 7, 2021 the entire contents of which is hereby incorporated by reference.

### BACKGROUND

Large-scale host cells transfections, e.g., for the manufacturing of recombinant adeno-associated virus (rAAV) gene therapies, is challenging. Such large-scale host cell transfection methods often fail to produce the quantity and quality of rAAV particles needed for clinical applications.

### SUMMARY

The present description encompasses, methods for transducing host cells with nucleic acids using an in-line complexer, and systems comprising an in-line complexer for transducing host cells. In some embodiments, methods and systems disclosed herein can be used to produce recombinant adeno-associated virus (rAAV) particles. Also disclosed herein are compositions comprising rAAV particles obtained from methods and systems disclosed herein, and uses of the same.

Disclosed herein is a method for transfecting host cells with nucleic acids, the method comprising: combining nucleic acids with a transfection reagent in an in-line complexer to form complexes that comprise the nucleic acids and the transfection reagent; and introducing the complexes into a vessel that comprises host cells under conditions that lead to transfection of the host cells with the nucleic acids, wherein the in-line complexer comprises (a) a first input tubing in communication at a proximal end to a source that comprises the nucleic acids, (b) a second input tubing in communication at a proximal end to a source that comprises the transfection reagent, and (c) an output tubing that is in communication: (i) at a proximal end to a distal end of the first input tubing and a distal end of the second input tubing; and (ii) at a distal end to the vessel that comprises the host cells, the first input tubing and the second input tubing are each in communication with a pump that has a flow rate of about 1 mL/min to 5000 mL/min, and the output tubing is about 60 mm to 100,000 mm in length and about 0.3 mm to 250 mm in inner diameter.

This disclosure also provides, a system for transfecting host cells with nucleic acids, the system comprising an in-line complexer and a vessel comprising host cells, wherein: the in-line complexer comprises (a) a first input tubing in communication at a proximal end to a source that comprises nucleic acids, (b) a second input tubing in communication at a proximal end to a source that comprises a transfection reagent, and (c) an output tubing that is in communication (i) at a proximal end to a distal end of the first input tubing and a distal end of the second input tubing and (ii) at a distal end to the vessel that comprises host cells, the first input tubing and the second input tubing are each in communication with a pump that has a flow rate of about 1 mL/min to about 5000 mL/min, and the output tubing is about 60 mm to 100,000 mm in length and about 0.3 mm to 250 mm in inner diameter.

In some embodiments, a system disclosed herein is used in a method for transfecting host cells with nucleic acids, said method comprising combining nucleic acids with a transfection reagent to form complexes that comprise the nucleic acids and the transfection reagent.

In some embodiments of any of the methods of transfecting host cells, or systems for transfecting host cells disclosed herein, the flow rate of the pump is about 10 mL/min to about 500 mL/min.

In some embodiments of any of the methods of transfecting host cells, or systems for transfecting host cells disclosed herein, an output tubing is about 60 mm to about 10,000 mm in length, for example about 3,000 mm to about 4,000 mm or about 6,000 mm to about 8,000 mm.

In some embodiments, an output tubing is about 3 mm to about 26 mm in inner diameter. In some embodiments, an output tubing is about 3.2 mm to about 25.4 mm in inner diameter, for example about 4 mm to about 8 mm, or about 20 mm to about 30 mm.

In some embodiments of any of the methods of transfecting host cells, or systems for transfecting host cells disclosed herein, a composition comprising nucleic acids in an amount of about 0.1% to about 10% of a vessel volume is introduced into a first tubing. In some embodiments, about 100 mL to about 150,000 mL of a composition comprising nucleic acids is introduced into a first tubing. In some embodiments, about 150 mL to about 100,000 mL of a composition comprising nucleic acids is introduced into a first tubing.

In some embodiments of any of the methods of transfecting host cells, or systems for transfecting host cells disclosed herein, a composition comprising a transfection reagent in an amount of about 0.1% to about 10% of a vessel volume is introduced into a second tubing. In some embodiments, about 100 mL to about 150,000 mL of a composition comprising a transfection reagent is introduced into a second tubing. In some embodiments, about 150 mL to about 100,000 mL of a composition comprising a transfection reagent is introduced into a second tubing.

In some embodiments of any of the methods of transfecting host cells, or systems for transfecting host cells disclosed herein, a composition comprising nucleic acids and a composition comprising a transfection reagent are introduced into a vessel (e.g., after being mixed in an output tubing) in an amount of about 0.1% to about 10% of a vessel volume.

In some embodiments, a combined volume of a composition comprising nucleic acids and a composition comprising a transfection reagent is about 0.1% to about 10% of a vessel volume.

In some embodiments, a combined volume of a composition comprising nucleic acids and a composition comprising a transfection reagent is about 100 mL to about 1,500,000 mL.

In some embodiments of any of the methods of transfecting host cells, or systems for transfecting host cells disclosed herein, wherein nucleic acids comprise one or more vectors. In some embodiments, the nucleic acids comprise one or more vectors encoding: (i) at least one payload flanked by an AAV inverted terminal repeat (ITR) on either side of the at least one payload, (ii) at least one AAV Rep polypeptide, (iii) at least one AAV Cap polypeptide, and/or (iv) at least one Adenoviral helper polypeptide.

In some embodiments, one or more vectors comprise: (i) a first vector encoding at least one payload flanked by an AAV ITR on either side of the at least one payload, (ii) a second vector encoding at least one AAV Rep polypeptide and at least one AAV Cap polypeptide, and/or (iii) a third vector encoding at least one Adenoviral helper polypeptide.

In some embodiments, one or more vectors comprise: (i) a first vector encoding at least one AAV Cap polypeptide and at least one payload flanked by an AAV ITR on either side of the at least one payload; and (ii) a second vector encoding at least one Adenoviral helper polypeptide and at least one AAV Rep polypeptide.

In some embodiments, the at least one Adenoviral helper polypeptide comprises one, two, three, or four of E1, E2A, E4orf6, or VA RNA polypeptides.

In some embodiments, an AAV Cap polypeptide comprises an AAV1, AAV2, AAV3A, AAB3B, AAV4, AAV5, AAV6, AAV7, AAV8, AAV9, AAVrh10, AAV-DJ, AAV-PHP.B, AAV-PHP.N, or AAV-PHP.S Cap polypeptide, or a variant of any of the foregoing. In some embodiments, an AAV Rep polypeptide comprises an AAV2 Rep polypeptide, or a variant thereof.

In some embodiments, an AAV ITR comprises an AAV2 ITR, or a variant thereof.

In some embodiments of any of the methods of transfecting host cells, or systems for transfecting host cells disclosed herein, the method further comprises culturing host cells under conditions suitable for producing recombinant AAV (rAAV) particles.

In some embodiments of any of the methods of transfecting host cells, or systems for transfecting host cells disclosed herein, the method further comprises collecting rAAV particles from a vessel. In some embodiments, AAV particles are collected without lysing host cells.

In some embodiments of any of the methods of transfecting host cells, or systems for transfecting host cells disclosed herein, nucleic acids are diluted in cell culture media.

In some embodiments of any of the methods of transfecting host cells, or systems for transfecting host cells disclosed herein, a transfection reagent comprises a polymer, or a lipid, or both. In some embodiments, a transfection reagent is or comprises a polymer. In some embodiments, a transfection reagent is or comprises a lipid. In some embodiments, a transfection reagent comprises a polymer and a lipid.

In some embodiments, a transfection reagent comprises polyethyleneimine (PEI), FectoVIR, TransIT-VirusGEN, or a combination thereof. In some embodiments, a transfection reagent is or comprises PEI.

In some embodiments of any of the methods of transfecting host cells, or systems for transfecting host cells disclosed herein, the complexes have an average diameter of about 100 nm to about 1000 nm. In some embodiments, the complexes have an average diameter of less than 700 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm.

In some embodiments, at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to about 1000 nm.

In some embodiments of any of the methods of transfecting host cells, or systems for transfecting host cells disclosed herein, it takes between about 30 seconds and about 3600 seconds for the nucleic acids, transfection reagent and/or complexes comprising the nucleic acid and transfection reagent to travel the length of the output tubing.

In some embodiments of any of the methods of transfecting host cells, or systems for transfecting host cells disclosed herein, it takes about 30 seconds to about 3600 seconds, for the nucleic acids, transfection reagent and/or complexes comprising the nucleic acid and transfection reagent to travel the length of the output tubing.

In some embodiments, it takes at least 30 seconds for the nucleic acids, transfection reagent and/or complexes comprising the nucleic acid and transfection reagent to travel the length of the output tubing.

In some embodiments, it takes no more than 3600 seconds for the nucleic acids, transfection reagent and/or complexes comprising the nucleic acid and transfection reagent to travel the length of the output tubing.

In some embodiments, it takes about 150 seconds for the nucleic acids, transfection reagent and/or complexes comprising the nucleic acid and transfection reagent to travel the length of the output tubing.

In some embodiments of any of the methods of transfecting host cells, or systems for transfecting host cells disclosed herein, an output tubing is configured in a coil configuration. In some embodiments, an output tubing is at an angle of at least 10 degrees relative to horizontal.

In some embodiments of any of the methods of transfecting host cells, or systems for transfecting host cells disclosed herein, a shear rate of a solution flowing through the first input tubing, the second input tubing and/or the output tubing is about 5 s⁻¹ to about 30 s⁻¹, for example about 10 s⁻¹ to about 20 s⁻¹, or about 12 s⁻¹ to about 18 s⁻¹.

In some embodiments of any of the methods of transfecting host cells, or systems for transfecting host cells disclosed herein, an in-line complexer further comprises one or more scales. In some embodiments, an in-line complexer comprises a scale attached to a source in communication with a first tubing. In some embodiments, an in-line complexer comprises a scale attached to a source in communication with a second tubing.

In some embodiments of any of the methods of transfecting host cells, or systems for transfecting host cells disclosed herein, an in-line complexer further comprises a mixer. In some embodiments, mixer is or comprises a static mixer. In some embodiments, a static mixer comprises a nozzle mixer, an injector, an orifice, a valve, a pump, or a combination thereof. In some embodiments, a static mixer is or comprise a nozzle mixer.

In some embodiments, a mixer is in communication with a distal end of a first and second input tubings and a proximal end of an output tubing. In some embodiments, an output tubing comprises an upstream and a downstream portion that are located upstream and downstream of a mixer.

In some embodiments of any of the methods of transfecting host cells, or systems for transfecting host cells disclosed herein, a vessel is a bioreactor. In some embodiments, a bioreactor comprises one or both of: (i) at least 1×10⁵ host cells; or (ii) at least 1 L of culture media.

In some embodiments of any of the methods of transfecting host cells, or systems for transfecting host cells disclosed herein, host cells are or comprise viable cells (vc).

In some embodiments of any of the methods of transfecting host cells, or systems for transfecting host cells disclosed herein, a bioreactor comprises one or both of: (i) about 1×10⁶ vc/mL to about 5×10⁶ vc/mL; or (ii) about 3 L to about 10,000 L culture media.

In some embodiments, a bioreactor is a continuous flow bioreactor, a batch process bioreactor, a perfusion bioreactor, or a fed batch bioreactor.

In some embodiments of any of the methods of transfecting host cells, or systems for transfecting host cells disclosed herein, host cells are suspension adapted host cells. In some embodiments, host cells are mammalian cells. In some embodiments, mammalian cells are HEK293 cells, CHO-K, or HeLa cells.

Also disclosed herein is a transfection complex produced by a method of transfecting host cells disclosed herein or a system of transfecting host cells disclosed herein.

Further disclosed herein is a culture comprising a plurality of host cells and a transfection complex disclosed herein.

This disclosure also provides a bioreactor comprising a culture comprising a plurality of host cells and a transfection complex disclosed herein. In some embodiments, a bioreactor comprises one or both of: (i) at least 1×10⁵ host cells; or (ii) at least 1 L of culture media. In some embodiments, host cells are or comprise viable cells (vc).

In some embodiments, a bioreactor comprises one or both of: (i) about 1×10⁶ vc/mL to about 5×10⁶ vc/mL; or (ii) about 3 L to about 10,000 L culture media.

In some embodiments, a bioreactor is selected from a continuous flow bioreactor, a batch process bioreactor, a perfusion bioreactor, and a fed batch bioreactor.

In some embodiments, a bioreactor is held under conditions suitable for formation of a plurality of rAAV particles.

This disclosure provides a composition comprising a plurality of rAAV particles produced by a method of transfecting host cells disclosed herein or using a system of transfecting host cells disclosed herein.

Further provided herein is a pharmaceutical composition comprising a plurality of rAAV particles produced by a method of transfecting host cells disclosed herein or using a system of transfecting host cells disclosed herein, and a pharmaceutically acceptable component.

Also provided herein are methods of administering a pharmaceutical composition disclosed herein to a subject. In some embodiments, a subject is a mammal. In some embodiments, a subject is a human.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Figures described below, which together make up the Drawing, are for illustration purposes only, not for limitation.

FIG. 1 is a graph depicting DNA/PEI nanoparticle size with respect to time as measured on a DLS instrument. Batch complexing of the DNA and PEI results in unstable complexes that aggregate over time. In-line complexing at a residence time of 5 minutes results in consistent production of nanoparticles over a longer period.

FIG. 2 is a graph showing comparable nanoparticle size for DNA/PEI complex formation between in-line complexing and traditional batch complexing at 5-minute incubation time post DNA and PEI mixture.

FIGS. 3A-3C depict cell culture performance using batch complexing of DNA and PEI (control) and the in-line complexing method for a triple plasmid transfection step. Cell viability (FIG. 3A) and viable cell density (FIG. 3B) were compared across the two conditions, demonstrating comparability. AAV titer (FIG. 3C; measured in viral genomes per mL using a ddPCR assay) was identical across the two conditions.

FIG. 4 is a graph showing a time course sampling for the in-line complexing method for particle size and AAV productivity (AAV genome titer) after addition into the cell culture. Particle size data shows a similar trend with what is observed in the batch complexing method of decreased complex size with decreased incubation time. The titer data displayed represents the average sample from 2 replicate shake flasks. A slight increase in productivity is shown with decreased complexing time.

FIG. 5 is a schematic of an exemplary in-line complexation process. In Step 1, plasmid DNA and transfection reagent (e.g., PEI) are diluted in separate containers. In Step 2, the plasmid DNA solution is pumped into a first tubing at a defined flow rate and the transfection reagent solution is pumped into a second tubing at a defined flow rate. In Step 3, the plasmid DNA solution and transfection reagent solution are pumped into an output tubing. The time both solutions spend moving through the tubing and forming a DNA-transfection reagent complex is the “complex time.” In Step 4, the solution with the DNA-transfection reagent is added to the bioreactor.

FIGS. 6A-6B depict consistent DNA-PEI nanoparticles produced for a 1000 L transfection with in-line complexation. FIG. 6A is a graph showing stable nanoparticles produced across the duration of the transfection process with in-line complexation for a 3 L bioreactor and a 1000 L bioreactor. The process used for the 3 L bioreactor had a 5 minute complex time and the process used for the 1000 L bioreactor has a 2.5 minute complex time. FIG. 6B shows the size of nanoparticles produced with inline complexation across experiments. The data shows increasing nanoparticle size (see y-axis) with increasing complex time (see x-axis).

FIGS. 7A-7B are graphs showing viral titer produced from transfection with in-line complexation in bioreactors of various sizes FIG. 7A shows similar AAV titer in a 3 L bioreactor, a 250 L bioreactor and 1000 L scale. FIG. 7B shows total AAV particles produced per batch in each bioreactor with the 1000 L bioreactor producing the highest amount of AAV particles.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

5′ and 3′: The terms “5′” and “3′” are relative terms to define a spatial relationship or directionality between two or more segment of a nucleic acid sequence. Thus, 3′ of a nucleic acid indicates a segment of the nucleic acid that is downstream of another segment, while 5′ indicates a segment of the nucleic acid that is upstream of another segment. For example, 3′ may indicate that a segment is in the 3′ half of the nucleic acid sequence or even at the 3′ end of the nucleic acid sequence. Similarly, 5′ may indicate that a segment is in the 3′ half of the nucleic acid sequence or even at the 5′ end of the nucleic acid sequence. Unless indicated otherwise, the directionality of a nucleic acid will be in the 5′ to 3′ direction of translation.

About or approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Adeno-associated virus (AA V): As used herein, the terms “Adeno-associated virus” and “AAV” refer to viral particles, in whole or in part, of the family Parvoviridae and the genus Dependoparvovirus. AAV is a small replication-defective, nonenveloped virus. AAV includes, but is not limited to, AAV serotype 1, AAV serotype 2, AAV serotype 3 (including serotypes 3A and 3B), AAV serotypes 4, AAV serotypes 5, AAV serotypes 6, AAV serotypes 7, AAV serotypes 8, AAV serotypes 9, AAV serotypes 10, AAV serotypes 11, AAV serotypes 12, AAV serotype 13, snake AAV, avian AAV, bovine AAV, canine AAV, equine AAV, ovine AAV, goat AAV, shrimp AAV, and any variant of any of the foregoing. Wild-type AAV is replication deficient and requires co-infection of cells by a helper virus, e.g., adenovirus, herpes, or vaccinia virus, e.g., an Ad2 or Ad5 virus, or supplementation of helper viral genes, in order to replicate.

Ad2 helper: As used herein, the term “Ad2 helper” refers to the Adenovirus serotype 2 (Ad2) helper virus (e.g., wildtype or recombinantly engineered Ad2 helper virus) and various Ad2 helper genes and/or Ad2 helper polypeptides, including, but not limited, to E1a, E1b, E2a, E4Orf6, VA RNA, and any variant or fragment of any of the foregoing. In some embodiments, an Ad2 helper vector (e.g., plasmid) encodes Ad2 helper polypeptides (e.g., one, two, three, or four of E1 (e.g., E1a and/or E1b), E2A, E4, or VA RNA) necessary to generate functional rAAV particles. In certain embodiments, the Ad2 helper vector is transfected into an E1 complementing cell line (e.g., HEK293). The nucleotide sequence of an Ad2 helper vector and Ad2 helper virus genes can be derived from the Adenovirus 2 genome (Genbank Accession No. J01917.1).

Ad5 helper: As used herein, the term “Ad5 helper” refers to the Adenovirus serotype 5 (Ad5) helper virus (e.g., wildtype or recombinantly engineered Ad5 helper virus) and various Ad5 helper genes and/or Ad5 helper polypeptides, including, but not limited, to E1a, E1b, E2a, E4Orf6, and/or VA RNA. In some embodiments, an Ad5 helper vector (e.g., plasmid) comprises Ad5 helper genes (e.g., one, two, three, or four of E1 (e.g., E1a and/or E1b), E2A, E4, or VA RNA) necessary to generation functional rAAV particles. In certain embodiments, the Ad5 helper vector is transfected into an E1 complementing cell line (e.g., HEK293). The nucleotide sequence of an Ad5 helper vector and Ad5 helper genes can be derived from the Adenovirus 5 genome (Genbank Accession No. AY601635).

Administration: As used herein, the term “administration” refers to the administration of a composition comprising rAAV particles as described herein to a subject. Administration may be by any appropriate route. For example, in some embodiments, administration may be local or systemic administration (e.g., to a mammal, e.g., to a human, e.g., a patient). A composition of the disclosure may be administered by injection or infusion by any route. For example, a composition may be administered by retinal, subretinal, intravitreal, suprachoroidal, intraspinal, intracisterna magna, or intrathecal injection or infusion. Additional exemplary routes of administration may include, but are not limited to, bronchial (e.g., bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (e.g., intratracheal instillation), transdermal, vaginal, and vitreal.

Bioreactor: The term “bioreactor,” as used herein, refers to any vessel used for the growth of a cell culture (e.g., a mammalian cell culture). The bioreactor can be of any size and/or any shape so long as it is useful for culturing a cell culture (e.g., a mammalian cell culture).

Cap polypeptide: As used herein, the term “Cap polypeptide” refers to the structural proteins that form a functional AAV capsid, which can in turn package DNA and infect a target cell. In some embodiments, Cap polypeptides will comprise all of the AAV capsid subunits, but less than all of the capsid subunits may be present as long as a functional capsid is produced. In some embodiments, the nucleic acid sequence encoding Cap polypeptides will be present on a single vector (e.g., plasmid). In some embodiments, the Cap polypeptide comprises an AAV1, AAV2, AAV3, AAV4, AAV5, AAV6, AAV7, AAV8, AAV9, AAV10, or AAV11 Cap polypeptide, or a variant of any of the foregoing. AAV capsid genes and proteins have been described in, e.g., Knipe et al., FIELDS VIROLOGY, Volume 1, (6th ed., Lippincott-Raven Publishers), which is hereby incorporated by reference in its entirety.

Cell Density: As used herein, the term “cell density” refers to that number of cells present in a given volume of medium or the number of cells present in a given surface area. For example, cell density may be represented as viable cells (vc)/cm² of culture medium or vc/mL.

Culture: As used herein, the terms “culture” and “cell culture” refer to a cell population (e.g., a eukaryotic cell population) that is suspended in or covered by a medium under conditions suitable to survival and/or growth of the cell population. As will be clear to those of ordinary skill in the art, these terms can also refer to the combination comprising the cell population and the medium.

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Gene: As used herein, the term “gene” refers to a DNA sequence that codes for a product (e.g., an RNA product and/or a polypeptide product). In some embodiments, a gene includes coding sequence (i.e., a sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory elements that, for example, may control or effect one or more aspects of gene expression (e.g., inducible expression, etc.).

Gene therapy: As used herein, the term “gene therapy” refers to insertion or deletion of specific genomic DNA sequences to treat or prevent a disorder or condition for which such therapy is sought. In some embodiments, the insertion or deletion of genomic DNA sequences occurs in specific cells (e.g., target cells). Target cells may be from a mammal and/or may be cells in a mammalian subject. Mammals include but are not limited to humans, dogs, cats, cows, sheep, pigs, llamas, etc. In some embodiments, heterologous DNA is transferred to target cells. The heterologous DNA may be introduced into the selected target cells in a manner such that the heterologous DNA is expressed and a therapeutic product encoded thereby is produced. Additionally or alternatively, the heterologous DNA may in some manner mediate expression of DNA that encodes the therapeutic product, or it may encode a product, such as a peptide or RNA that in some manner mediates or modulates, directly or indirectly, expression of a therapeutic product. Genetic therapy may also be used to deliver nucleic acid encoding a gene product that replaces a defective gene or supplements a gene product produced by the mammal or the cell in which it is introduced. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression. Such an inhibitor or repressor or other modulator can be a polypeptide, peptide, or nucleic acid (e.g., DNA or RNA). Gene therapy may include in vivo or ex vivo techniques. In some embodiments, viral and non-viral based gene transfer methods can be used to introduce a nucleic acid encoding a polypeptide of interest or to introduce a therapeutic nucleic acid into mammalian cells or target tissues. Non-viral vector delivery systems include DNA plasmids, naked nucleic acid, and nucleic acid complexed with a delivery vehicle, such as poloxamers or liposomes. Viral vector delivery systems include DNA and RNA viruses, which have either episomal or integrated genomes after delivery to the cell. For a review of gene therapy procedures, see Anderson, Science 256:808-813 (1992); Miller, Nature 357:455-460 (1992); Feuerbach et al., Kidney International 49:1791-1794 (1996); Urnov et al., Nature Reviews Genetics 11, 636-646 (2010); and Collins et al., Proceedings Biological Sciences/The Royal Society, 282(1821):pii 20143003 (2015), each of which is hereby incorporated by reference in its entirety.

Host Cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence).

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

In-line complexer: An “in-line complexer” as used herein refers to a system through which at least two components to be contacted with one another are passed. In some embodiments, an in-line complexer comprises at least one, two or three tubings. In some embodiments, an in-line complexer is in communication with a vessel, e.g., comprising host cells. In some embodiments, an in-line complexer comprises a first input tubing in communication at a proximal end to a source, e.g., a first source, e.g., comprising nucleic acids. In some embodiments, an in-line complexer comprises a second input tubing in communication at a proximal end to a source, e.g., a second source, e.g., comprising a transfection reagent. In some embodiments, an in-line complexer comprises an output tubing that is in communication: (i) at a proximal end to a distal end of a first input tubing and a distal end of a second input tubing; and (ii) at a distal end to a vessel that comprises host cells. In some embodiments, a first input tubing and a second input tubing are each in communication with a pump, e.g., having a flow rate described herein. In some embodiments, a pump can be used to control a flow rate of one or more components in a tubing. In some embodiments, a length of a tubing and/or an inner diameter of a tubing can be adjusted to control the amount of time a liquid and/or a complex spends in a tubing. In some embodiments, an in-line complexer further comprises a mixer, e.g., a static mixer. In some embodiments, a mixer is in communication with a distal end of a first and second input tubings and a proximal end of an output tubing. In some embodiments, an in-line complexer is used to contact components from a first source (e.g., comprising nucleic acids) with components from a second source (e.g., comprising a transfection reagent). In some embodiments, contacting components from a first source with components from a second source in an in-line complexer forms a complex comprising components from the first source (e.g., comprising nucleic acids) and components from the second source (e.g., comprising transfection reagent).

“Improve,” “increase,” “inhibit,” or “reduce”: As used herein the terms “improve”, “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single sample, e.g., of a culture medium) under otherwise comparable conditions absent presence of (e.g., prior to and/or after) a particular agent or treatment, or in presence of an appropriate comparable reference agent. In some embodiments, an appropriate reference measurement may be or comprise a measurement in a comparable system known or expected to respond in a particular way, in presence of the relevant agent or treatment.

Medium: As used herein, the terms “medium,” “culture medium,” and “growth medium” refer to a solution comprising nutrients to nourish cells (e.g., growing cells, e.g., eukaryotic cells). Typically, these solutions provide essential and non-essential amino acids, vitamins, energy sources, lipids, and trace elements required by the cell for survival and/or minimal growth. The solution can also comprise components that enhance survival and/or growth above the minimal rate, including hormones and growth factors. The solution can be formulated to a pH and concentration of one or more salts that are optimal for cellular survival and/or proliferation. For example, the medium can also be a “defined medium” or “chemically defined medium,” e.g., a serum-free medium that contains no proteins, hydrolysates, or components of unknown composition. Defined media are free of animal-derived components and all components have a known chemical structure. One of skill in the art understands a defined medium can comprise recombinant polypeptides, for example, but not limited to, hormones, cytokines, interleukins, and/or other signaling molecules.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly—refers to a nucleic acid containing 2 to about 10,000, 2 to about 50,000, or 2 to about 100,000 nucleotide monomer units. In some embodiments, the prefix oligo—refers to a nucleic acid containing 2 to about 200 nucleotide monomer units. In accordance with the methods and compositions described herein, in some embodiments, an RNA comprises a short hairpin RNA (shRNA), small interfering RNA (siRNA), mRNA, snRNA, CRISPR/Cas guide RNA, microRNA (miRNA), and/or a precursor thereof.

Payload: As used herein, the term “payload” refers to a nucleic acid sequence of interest (e.g., comprising a sequence that encodes a target payload, such as a target polypeptide) that is desired to be introduced into a cell, tissue, organ, organism, and/or system comprising cells. A target payload can be a heterologous protein with a therapeutic purpose, e.g., an enzyme or antibody. The target payload can be a heterologous nucleic acid with a therapeutic purpose, e.g., an miRNA, siRNA, shRNA, mRNA, snRNA, or CRISPR/Cas guide RNA, or a precursor thereof. One of skill in the art will recognize that the target payload can be selected from any heterologous protein or nucleic acid of interest. As used herein, “encode” or “encodes” means directs the expression of or processed into. For example, as used herein, a nucleic acid encodes a polypeptide sequence if it directs the expression of that polypeptide sequence. As another example, as used herein, a nucleic acid precursor (e.g., a pri-miRNA or pre-miRNA) encodes a further processed version of the nucleic acid (e.g., mature miRNA) if it is processed into the further processed version.

Pharmaceutical composition: As used herein, the term “pharmaceutical composition” refers to a composition comprising rAAV particles that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to achieve a desired therapeutic effect when administered to a subject or population in need thereof (e.g., by a statistically significant probability). A pharmaceutical composition may be specially formulated for administration in solid or liquid form. In some embodiments, a pharmaceutical composition is formulated for administration by parenteral administration, such as by subcutaneous, intramuscular, intravenous or epidural injection. In some embodiments, a pharmaceutical composition is formulated as a sterile solution or suspension, e.g., in a sustained-release formulation. Pharmaceutical compositions of the disclosure may be formulated for administration by injection or infusion (e.g., subcutaneous, intramuscular, intravenous or epidural injection or infusion). For example, compositions may be formulated for administration by retinal, subretinal, intravitreal, suprachoroidal, intraspinal, intracisterna magna, or intrathecal injection or infusion. In some embodiments, a pharmaceutical composition is intended and suitable for administration to a human subject. In some embodiments, a pharmaceutical composition is substantially free of contaminants (e.g., sterile and substantially pyrogen-free). Formulations of the pharmaceutical compositions may include, but are not limited to, formulations for oral administration, such as drenches (aqueous or non-aqueous solutions or suspensions), tablets (e.g., targeted for buccal, sublingual, and systemic absorption), boluses, powders, granules, pastes for application to the tongue; topical application, such as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (e.g., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Recombinant AAV(rAAV)particle: A “recombinant AAV particle”, or “rAAV particle,” as used herein, refers to an infectious, replication-defective viral particle comprising an AAV protein shell encapsulating a payload that is flanked on both sides by ITRs. An AAV particle is produced in a suitable host cell (e.g., a HEK293 cell). For example, the host cell is transfected with at least one vector encoding one or more helper polypeptides (e.g., Ad2 helper polypeptides), at least one Rep polypeptide, at least one Cap polypeptide, and at least one payload (e.g., for polypeptide expression or a therapeutic nucleic acid), such that the host cell is capable of producing the Rep and Cap polypeptides necessary for packing the rAAV particle. rAAV particles may be used for subsequent gene delivery.

Rep polypeptide: The term “Rep polypeptide”, as used herein, refers to the AAV non-structural proteins that mediate AAV replication for the production of AAV particles. The AAV replication genes and proteins have been described in, e.g., Knipe et al., FIELDS VIROLOGY, Volume 1, (6th ed., Lippincott-Raven Publishers), which is hereby incorporated by reference in its entirety.

Seeding: The term “seeding” as used herein refers to the process of providing a cell culture to a vessel (e.g., a bioreactor or culture flask). For example, the process of providing a cell culture may include propagation of the cells in another bioreactor or vessel before providing to the bioreactor or other vessel. The cells have been frozen and thawed immediately prior to providing them to the bioreactor or vessel. The term “seeding” refers to providing any number of cells, including a single cell.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein, e.g., a neurological disease or disorder or a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Titer: As used herein, the term “titer” refers to the quantity of virus in a given volume. Titer, for example, can be expressed as viral genome copies (vg) per given volume or plaque forming units (pfu) per given volume. In some embodiments, titer can be expressed as number of capsids per given volume.

Transfection: As used herein, the term “transfection” refers to the introduction of nucleic acid molecules, such as DNA or RNA (e.g. mRNA) molecules, into cells, such as eukaryotic cells (e.g., mammalian cells). For example, transfection can include vector-based transfection, viral-based transfection, electroporation, lipofection (e.g., with cationic lipids and/or liposomes), calcium phosphate precipitation, nanoparticle-based transfection, and/or transfection based on cationic polymers (e.g., DEAE-dextran or polyethylenimine).

Treating: As used herein, the term “treating” refers to providing treatment, e.g., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a condition, disease, or disorder, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition comprising rAAV particles of the disclosure can be administered to a subject who has developed a disorder or is at increased risk of developing such a disorder relative to a member of the general population. A composition of the disclosure can be administered prophylactically or before development of any symptom or manifestation of the condition. Typically, in this case, the subject will be at risk of developing the condition.

Vector: As used herein, the term “vector” refers to a molecule comprising a nucleic acid molecule, where the vector is capable of transporting the nucleic acid molecule into a cell. By way of non-limiting example, one type of vector is a “plasmid,” which refers to a circular double stranded DNA loop into which additional DNA segments may be ligated. Another type of vector is a viral vector, wherein additional DNA segments may be packaged into a viral capsid and can be transferred into another cell and/or organism. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference in its entirety.

### DETAILED DESCRIPTION OF CERTAIN EMBODIMENTS

The present disclosure provides, inter alia, improved methods for transducing host cells with nucleic acids using an in-line complexer and systems comprising an in-line complexer for transducing host cells. Typically, host cells are transfected with nucleic acids by first combining the nucleic acids with a transfection reagent to generate a complex comprising the nucleic acids and the transfection reagent which is subsequently introduced to host cells. The complex comprising nucleic acids and transfection reagent facilitates movement of the nucleic acids into host cells. One main challenge with this method is that the transfection efficiency of complexes comprising nucleic acids and transfection reagent (also referred to herein in certain embodiments as “nanoparticles”) is time-dependent with peak transfection efficiency at about 5 minutes after the nucleic acid and transfection reagent have been contacted with each other. Increasing the amount of time the nucleic acid and transfection reagent are in contact, increases aggregation of the complexes which leads to a reduction in transfection efficiency. This instability poses a problem in large-scale manufacturing processes where the volumes of the nucleic acid and transfection reagent are both large and completing the entire operation in a short amount of time, e.g., 5 minutes, can be technically challenging.

The present disclosure is based, in part, on the discovery that by complexing nucleic acids and a transfection reagent using an in-line mixing device, a pre-determined hold time can be achieved, e.g., through the length of the device. Furthermore, disclosed transfection methods also allow for the nucleic acid and transfection reagent residence time, as well as the size of the complex comprising nucleic acids and transfection reagent to be controlled. This allows for host cell transfections with controlled addition of the complex comprising nucleic acids and transfection reagent into a vessel, e.g., a bioreactor. The transfection methods disclosed herein also enable automated, standardized, reproducible and/or scalable host cell transfections. Accordingly, the present disclosure provides, inter alia, host cell transfection methods and systems for transducing host cells which can be used in recombinant adeno-associated virus (rAAV) manufacturing processes to efficiently and reproducibly generate high titer, high purity, and/or potent quantities of rAAV particles at large scales.

Without wishing to be bound by theory, in some embodiments, the use of a host cell transfection method comprising an in-line complexer as described herein leads to production of rAAV particles having improved characteristics and/or leads to more scalable manufacturing methods for rAAV particles, relative to a host cell transfection method using standard batch transfection techniques.

In certain embodiments, a method for transfecting host cells comprising combining nucleic acids with a transfection reagent in an in-line complexer forms complexes comprising the nucleic acids and the transfection reagent. In some embodiments, the complexes formed using a method or system disclosed herein have an average diameter of about 100 nm to about 1000 nm. In some embodiments, the complexes have an average diameter of less than 700 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm.

In some embodiments, the amount of time a nucleic acid is in contact with a transfection reagent can be controlled by, e.g., modulating a flow rate of a tubing, modulating a length of a tubing, and/or modulating an inner diameter of a tubing. In some embodiments of a method or system disclosed herein, a nucleic acid and a transfection reagent are in contact for a duration of time that a nucleic acid and a transfection reagent travel a length of a tubing. In some embodiments, it takes between about 30 seconds to about 600 seconds for a nucleic acid, a transfection reagent and/or complexes comprising a nucleic acid and a transfection reagent to travel a length of a tubing.

### In-Line Complexer

In-line mixers are known in the art and used in a variety of fields, such as in the food industry. Typically, an in-line mixer is made up of two tubings or inlets in a Y-shaped configuration which join at an intersection to form a single output tubing or outlet. See, e.g., U.S. Pat. No. 6,534,483 which discloses in-line mixers and uses of the same. Notably, while in-line mixers are known in the art, the present disclosure is the first to describe the use of an in-line complexer (which shares certain characteristics with an in-line mixer) to transfect host cells for the production of rAAV particles. Furthermore, the present disclosure also discloses the important utility of using in-line mixers for large-scale manufacturing of rAAV particles—a process which is known to be technically challenging.

In some embodiments, an in-line complexer disclosed herein comprises at least one tubing. In some embodiments, an in-line complexer disclosed herein comprises at least two tubings. In some embodiments, an in-line complexer disclosed herein comprises three tubings. In some embodiments, an in-line complexer disclosed herein comprises a first input tubing, a second input tubing and a third tubing, e.g., an output tubing.

In some embodiments of an in-line complexer comprising a first input tubing, a second input tubing and a third tubing (e.g., an output tubing), all three tubings are connected to each other with one or more connectors. In some embodiments, all three tubings are connected to each other with one connector. In some embodiments, the connector has a “Y” shape. In some embodiments, all three tubings are connected to each other with one connector having a “Y” shape. In some embodiments, all three tubings are connected to each other in a “Y” configuration.

In some embodiments of an in-line complexer comprising a first input tubing, a second input tubing and a third tubing (e.g., an output tubing), all three tubings are made of a single material. In some embodiments, the first tubing, second tubing and third tubing are connected to each other without any connectors. In some embodiments, the first tubing, second tubing and third tubing are made of a single material and are configured in a “Y” configuration.

In some embodiments, an in-line complexer disclosed herein comprises: (a) a first input tubing in communication at a proximal end to a source that comprises nucleic acids, (b) a second input tubing in communication at a proximal end to a source that comprises a transfection reagent, and (c) an output tubing that is in communication: (i) at a proximal end to a distal end of the first input tubing and a distal end of the second input tubing; and (ii) at a distal end to a vessel comprising host cells.

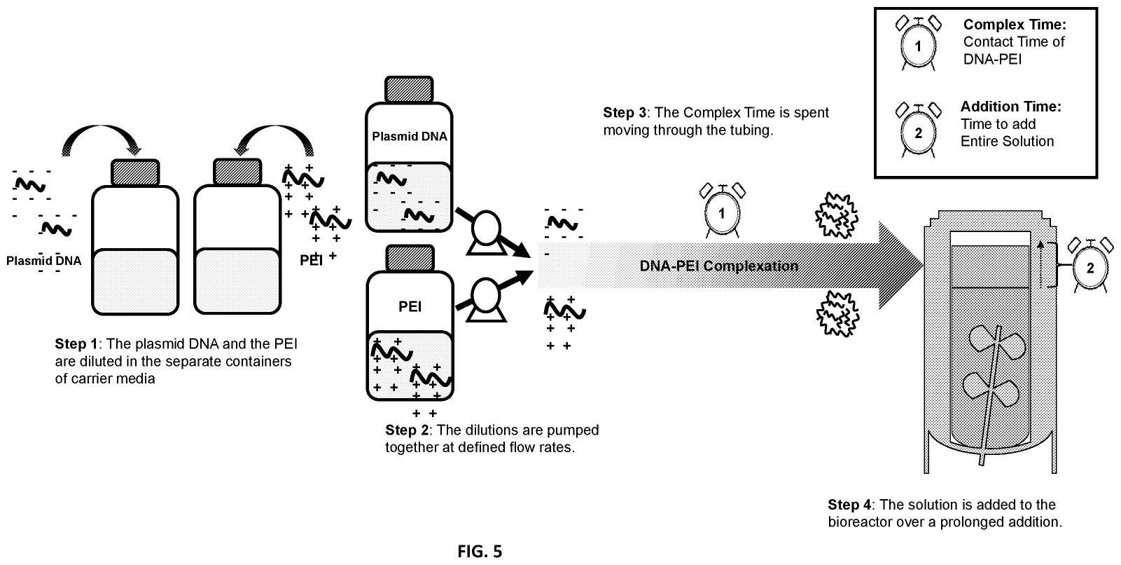

An exemplary in-line complexer and an in-line complexation process are disclosed in FIG. 5. In some embodiments, an in-line complexation process comprises a step in which nucleic acids and a transfection reagent are diluted in separate containers (se Step 1 in FIG. 5). In some embodiments, an in-line complexation process comprises a step in which a composition comprising nucleic acids is pumped into a first tubing at a defined flow rate and/or a composition comprising a transfection reagent solution is pumped into a second tubing at a defined flow rate (See Step 2 in FIG. 5). In some embodiments, an in-line complexation process comprises a step in which a composition comprising nucleic acids and a composition comprising a transfection reagent are pumped into an output tubing (See Step 3 in FIG. 5). In some embodiments, an in-line complexation process comprises a step in which a complex comprising nucleic acids and a transfection reagent is added to a vessel, e.g., a bioreactor (See Step 4 in FIG. 5).

In some embodiments, a composition (e.g., a composition comprising nucleic acids) in a first input tubing and a composition (e.g., a composition comprising a transfection reagent) in a second tubing are mixed (e.g., combined) in an output tubing. In some embodiments, mixing of a composition (e.g., a composition comprising nucleic acids) in a first input tubing with a composition (e.g., a composition comprising a transfection reagent) in a second tubing occurs as each composition enters an output tubing or travels a length of an output tubing.

In some embodiments, mixing of a composition (e.g., a composition comprising nucleic acids) in a first input tubing with a composition (e.g., a composition comprising a transfection reagent) in a second tubing, in an output tubing results in formation of a complex between a composition in a first tubing and a composition in a second tubing.

In some embodiments, mixing of a composition (e.g., a composition comprising nucleic acids) in a first input tubing with a composition (e.g., a composition comprising a transfection reagent) in a second tubing in an output tubing involves one or more priming steps (i.e., steps of removing air bubbles in one or more of the tubings).

In some embodiments, the one or more priming steps are done manually.

In some embodiments, the system is capable of self-priming. In some embodiments, self-priming comprises removal of air bubbles such that a composition (e.g., a composition comprising nucleic acids) in a first input tubing can mix with (e.g., form a complex with) a composition (e.g., a composition comprising a transfection reagent) in a second tubing. In some embodiments, self-priming comprises an output tubing configured in a coil configuration. In some embodiments, self-priming comprises an output tubing (e.g., in a coil configuration) at an angle that allows removal of air bubbles. In some embodiments, an output tubing (e.g., in a coil configuration) is at an angle of at least 10 degrees relative to horizontal.

In some embodiments of an in-line complexer disclosed herein, a solution flowing through one or more tubings of an in-line complexer has a pre-determined shear rate. In some embodiments, a shear rate of a solution flowing through a first input tubing, a second tubing, and/or an output tubing is about 5 s⁻¹ to about 30 s⁻¹, about 6 s⁻¹ to about 30 s⁻¹, about 7 s⁻¹ to about 30 s⁻¹, about 8 s⁻¹ to about 30 s⁻¹, about 9 s⁻¹ to about 30 s⁻¹, about 10 s⁻¹ to about 30 s⁻¹, about 11 s⁻¹ to about 30 s⁻¹, about 12 s⁻¹ to about 30 s⁻¹, about 13 s⁻¹ to about 30 s⁻¹, about 14 s⁻¹ to about 30 s⁻¹, about 15 s⁻¹ to about 30 s⁻¹, about 16 s⁻¹ to about 30 s⁻¹, about 17 s⁻¹ to about 30 s⁻¹, about 18 s⁻¹ to about 30 s⁻¹, about 19 s⁻¹ to about 30 s⁻¹, about 20 s⁻¹ to about 30 s⁻¹, about 21 s⁻¹ to about 30 s⁻¹, about 22 s⁻¹ to about 30 s⁻¹, about 23 s⁻¹ to about 30 s⁻¹, about 24 s⁻¹ to about 30 s⁻¹, about 25 s⁻¹ to about 30 s⁻¹, about 26 s⁻¹ to about 30 s⁻¹, about 27 s⁻¹ to about 30 s⁻¹, about 28 s⁻¹ to about 30 s⁻¹, about 29 s⁻¹ to about 30 s⁻¹, about 5 s⁻¹ to about 29 s⁻¹, about 5 s⁻¹ to about 28 s⁻¹, about 5 s⁻¹ to about 27 s⁻¹, about 5 s⁻¹ to about 26 s⁻¹, about 5 s⁻¹ to about 25 s⁻¹, about 5 s⁻¹ to about 24 s⁻¹, about 5 s⁻¹ to about 23 s⁻¹, about 5 s⁻¹ to about 22 s⁻¹, about 5 s⁻¹ to about 21 s⁻¹, about 5 s⁻¹ to about 20 s⁻¹, about 5 s⁻¹ to about 19 s⁻¹, about 5 s⁻¹ to about 18 s⁻¹, about 5 s⁻¹ to about 17 s⁻¹, about 5 s⁻¹ to about 16 s⁻¹, about 5 s⁻¹ to about 15 s⁻¹, about 5 s⁻¹ to about 14 s⁻¹, about 5 s⁻¹ to about 13 s⁻¹, about 5 s⁻¹ to about 12 s⁻¹, about 5 s⁻¹ to about 11 s⁻¹, about 5 s⁻¹ to about 10 s⁻¹, about 5 s⁻¹ to about 9 s⁻¹, about 5 s⁻¹ to about 8 s⁻¹, about 5 s⁻¹ to about 7 s⁻¹, about 5 s⁻¹ to about 6 s⁻¹.

In some embodiments, a shear rate of a solution flowing through a first input tubing, a second tubing, and/or an output tubing is about 5 s⁻¹, about 6 s-K, about 7 s⁻¹, about 8 s⁻¹, about 9 s⁻¹, about 10 s⁻¹, about 11 s⁻¹, about 12 s⁻¹, about 13 s⁻¹, about 14 s⁻¹, about 15 s⁻¹, about 16 s⁻¹, about 17 s⁻¹, about 18 s⁻¹, about 19 s⁻¹, about 20 s⁻¹, about 21 s⁻¹, about 22 s⁻¹, about 23 s⁻¹, about 24 s⁻¹, about 25 s⁻¹, about 26 s⁻¹, about 27 s⁻¹, about 28 s⁻¹, about 29 s⁻¹, about 30 s⁻¹.

In some embodiments, an in-line complexer disclosed herein can be scaled for use with a bioreactor, e.g., a bioreactor comprising about 1 L to about 10,000 L. In some embodiments, scaling of an in-line complexer disclosed herein comprises maintaining at least one parameter constant while adjusting one or more other parameters. In some embodiments, a parameter that is kept constant is a shear rate. In some embodiments, a parameter that is kept constant is a complex time. In some embodiments, a shear rate and complex time are kept constant.

In some embodiments, an in-line complexer disclosed herein further comprises a mixer. In some embodiments, a mixer is or comprises a static mixer. In some embodiments, a static mixer comprises a nozzle mixer, an injector, an orifice, a valve, a pump, or a combination thereof. In some embodiments, astatic mixer is or comprise a nozzle mixer.

In some embodiments, an in-line complexer disclosed herein further comprises one or more scales. In some embodiments, a scale can be attached to a source in communication with a first tubing (e.g., a source comprising nucleic acids, e.g., DNA). In some embodiments, a scale can be attached to a source in communication with a second tubing (e.g., a source comprising a transfection reagent, e.g., PEI). In some embodiments, a scale can be further connected to a pump connected to a first tubing. In some embodiments, a scale can be further connected to a pump connected to a second tubing. In some embodiments, information obtained from a scale can be used to control a flow rate, e.g., a flow rate of a first tubing and/or a flow rate of a second tubing.

In some embodiments, an in-line complexer disclosed herein can be used with a bioreactor comprising one or more probes. In some embodiments, one or more probes are biocapacitance probes. In some embodiments, a biocapacitance probe can detect cell density. In some embodiments, a biocapacitance probe allows for transfection of cells with an in-line complexer at a pre-determined cell density.

In some embodiments, an in-line complexer disclosed herein can be used with a bioreactor comprising one or more scales. In some embodiments, a bioreactor scale allows for addition of a pre-determined transfection volume. In some embodiments, a bioreactor scale allows for the addition of a transfection volume to be controlled and/or monitored.

### First and Second Input Tubings

In some embodiments, an in-line complexer disclosed herein comprises a first input tubing in communication at a proximal end to a source that comprises nucleic acids. In some embodiments, an in-line complexer disclosed herein comprises a second input tubing in communication at a proximal end to a source that comprises a transfection reagent.

In some embodiments, a first input tubing and a second input tubing are each in communication with a pump. In some embodiments, a first input tubing and a second input tubing are both in communication with a single pump (e.g., one pump in communication with both tubings).

In some embodiments, a first input tubing and a second input tubing are each in communication with two separate pumps. In some embodiments, the two separate pumps can be operated independently.

In some embodiments, a pump has a flow rate of about 1 mL/min, about 10 mL/min, about 20 mL/min, about 30 mL/min, about 40 mL/min, about 50 mL/min, about 60 mL/min, about 70 mL/min, about 80 mL/min, about 90 mL/min, about 100 mL/min, about 200 mL/min, about 300 mL/min, about 400 mL/min, about 500 mL/min, about 600 mL/min, about 700 mL/min, about 800 mL/min, about 900 mL/min, about 1000 mL/min, about 2000 mL/min, about 3000 mL/min, about 4000 mL/min, or about 5000 mL/min.

In some embodiments, a pump has a flow rate of about 1 mL/min to about 5000 mL/min, about 1 mL/min to about 4000 mL/min, about 1 mL/min to about 3000 mL/min, about 1 mL/min to about 2000 mL/min, about 1 mL/min to about 1000 mL/min, about 1 mL/min to about 500 mL/min, about 1 mL/min to about 400 mL/min, about 1 mL/min to about 300 mL/min, about 1 mL/min to about 200 mL/min, about 1 mL/min to about 100 mL/min, about 1 mL/min to about 50 mL/min, about 1 mL/min to about 10 mL/min, about 10 mL/min to about 5000 mL/min, 20 mL/min to about 5000 mL/min, 30 mL/min to about 5000 mL/min, 40 mL/min to about 5000 mL/min, 50 mL/min to about 5000 mL/min, 100 mL/min to about 5000 mL/min, 200 mL/min to about 5000 mL/min, 300 mL/min to about 5000 mL/min, 400 mL/min to about 5000 mL/min, 500 mL/min to about 5000 mL/min, 1000 mL/min to about 5000 mL/min, 2000 mL/min to about 5000 mL/min, 3000 mL/min to about 5000 mL/min, 4000 mL/min to about 5000 mL/min.

In some embodiments, a pump has a flow rate of about 10 mL/min to about 500 mL/min.

### Output Tubing

In some embodiments, an in-line complexer disclosed herein comprises a third tubing, e.g., an output tubing. In some embodiments, an output tubing is in communication: (i) at a proximal end to a distal end of a first input tubing and a distal end of a second input tubing; and (ii) at a distal end to a vessel comprising host cells.

In some embodiments, an output tubing has a length of about 60 mm, about 70 mm, about 80 mm, about 90 mm, about 100 mm, about 200 mm, about 300 mm, about 400 mm, about 500 mm, about 600 mm, about 700 mm, about 800 mm, about 900 mm, about 1000 mm, about 2000 mm, about 5000 mm, about 10,000 mm, about 20,000 mm, about 30,000 mm, about 40,000 mm, about 50,000 mm, or about 100,000 mm.

In some embodiments, an output tubing has a length of 60 mm+/−10%, 70 mm+/−10%, 80 mm+/−10%, 90 mm+/−10%, 100 mm+/−10%, 200 mm+/−10%, 300 mm+/−10%, 400 mm+/−10%, 500 mm+/−10%, 600 mm+/−10%, 700 mm+/−10%, 800 mm+/−10%, 900 mm+/−10%, 1000 mm+/−10%, 2000 mm+/−10%, 5000 mm+/−10%, 10,000 mm+/−10%, 20,000 mm+/−10%, 30,000 mm+/−10%, 40,000 mm+/−10%, 50,000 mm+/−10%, or 100,000 mm+/−10%.

In some embodiments, an output tubing has a length of 2,000 mm+/−10%.

In some embodiments, an output tubing has a length of 3,500 mm+/−10%.

In some embodiments, an output tubing has a length of 4,000 mm+/−10%.

In some embodiments, an output tubing has a length of 5,000 mm+/−10%.

In some embodiments, an output tubing has a length of 6,000 mm+/−10%.

In some embodiments, an output tubing has a length of 7,500 mm+/−10%.

In some embodiments, an output tubing has a length of about 60 mm to about 100,000 mm, about 100 mm to about 100,000 mm, about 200 mm to about 100,000 mm, 300 mm to about 100,000 mm, 400 mm to about 100,000 mm, 500 mm to about 100,000 mm, 600 mm to about 100,000 mm, 700 mm to about 100,000 mm, 800 mm to about 100,000 mm, 900 mm to about 100,000 mm, 1000 mm to about 100,000 mm, 5000 mm to about 100,000 mm, 10,000 mm to about 100,000 mm, 50,000 mm to about 100,000 mm, about 60 mm to about 50,000 mm, about 60 mm to about 10,000 mm, about 60 mm to about 5000 mm, about 60 mm to about 1000 mm, about 60 mm to about 900 mm, about 60 mm to about 800 mm, about 60 mm to about 700 mm, about 60 mm to about 600 mm, about 60 mm to about 500 mm, about 60 mm to about 400 mm, about 60 mm to about 300 mm, about 60 mm to about 200 mm, about 60 mm to about 100 mm.

In some embodiments, an output tubing has a length of about 60 mm to about 10,000 mm.

In some embodiments, an output tubing has an inner diameter of about 0.3 mm, about 0.6 mm, about 1 mm, about 2 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 4 mm, about 5 mm, about 10 mm, about 15 mm, about 20 mm, about 21 mm, about 22 mm, about 23 mm, about 24 mm, about 25 mm, about 25.1 mm, about 25.2 mm, about 25.3 mm, about 25.4 mm, about 25.5 mm, about 25.6 mm, about 25.7 mm, about 25.8 mm, about 25.9 mm, about 26 mm, about 30 mm, about 40 mm, about 50 mm, about 60 mm, about 70 mm, about 80 mm, about 90 mm, about 100 mm, about 150 mm, about 200 mm, about 250 mm.

In some embodiments, an output tubing has an inner diameter of about 0.3 mm to about 250 mm, about 0.6 mm to about 250 mm, about 1 mm to about 350 mm, about 2 mm to about 250 mm, about 2.5 mm to about 250 mm, about 2.6 mm to about 250 mm, about 2.7 mm to about 250 mm, about 2.8 mm to about 250 mm, about 2.9 mm to about 250 mm, about 3 mm to about 250 mm, about 3.1 mm to about 250 mm, about 3.2 mm to about 250 mm, about 3.3 mm to about 250 mm, about 3.4 mm to about 250 mm, about 3.5 mm to about 250 mm, about 4 mm to about 250 mm, about 5 mm to about 250 mm, about 10 mm to about 250 mm, about 15 mm to about 250 mm, about 20 mm to about 250 mm, about 21 mm to about 250 mm, about 22 mm to about 250 mm, about 23 mm to about 250 mm, about 24 mm to about 250 mm, about 25 mm to about 250 mm, about 25.1 mm to about 250 mm, about 25.2 mm to about 250 mm, about 25.3 mm to about 250 mm, about 25.4 mm to about 250 mm, about 25.5 mm to about 250 mm, about 25.6 mm to about 250 mm, about 25.7 mm to about 250 mm, about 25.8 mm to about 250 mm, about 25.9 mm to about 250 mm, about 26 mm to about 250 mm, about 30 mm to about 250 mm, about 40 mm to about 250 mm, about 50 mm to about 250 mm, about 60 mm to about 250 mm, about 70 mm to about 250 mm, about 80 mm to about 250 mm, about 90 mm to about 250 mm, about 100 mm to about 250 mm, about 150 mm to about 250 mm, about 200 mm to about 250 mm.

In some embodiments, an output tubing has an inner diameter of about 0.3 mm to about 200 mm, about 0.3 mm to about 150 mm, about 0.3 mm to about 100 mm, about 0.3 mm to about 90 mm, about 0.3 mm to about 80 mm, about 0.3 mm to about 70 mm, about 0.3 mm to about 60 mm, about 0.3 mm to about 50 mm, about 0.3 mm to about 40 mm, about 0.3 mm to about 30 mm, about 0.3 mm to about 26 mm, about 0.3 mm to about 25.9 mm, about 0.3 mm to about 25.8 mm, about 0.3 mm to about 25.7 mm, about 0.3 mm to about 25.6 mm, about 0.3 mm to about 25.5 mm, about 0.3 mm to about 25.4 mm, about 0.3 mm to about 25.3 mm, about 0.3 mm to about 25.2 mm, about 0.3 mm to about 25.1 mm, about 0.3 mm to about 25 mm, about 0.3 mm to about 24 mm, about 0.3 mm to about 23 mm, about 0.3 mm to about 22 mm, about 0.3 mm to about 21 mm, about 0.3 mm to about 20 mm, about 0.3 mm to about 15 mm, about 0.3 mm to about 10 mm, about 0.3 mm to about 5 mm, about 0.3 mm to about 4 mm, about 0.3 mm to about 3.5 mm, about 0.3 mm to about 3.4 mm, about 0.3 mm to about 3.3 mm, about 0.3 mm to about 3.2 mm, about 0.3 mm to about 3.1 mm, about 0.3 mm to about 3 mm, about 0.3 mm to about 2.9 mm, about 0.3 mm to about 2.8 mm, about 0.3 mm to about 2.7 mm, about 0.3 mm to about 2.6 mm, about 0.3 mm to about 2.5 mm, about 0.3 mm to about 2 mm, about 0.3 mm to about 1 mm, about 0.3 mm, or about 0.3 mm to about 0.6 mm.

In some embodiments, an output tubing has an inner diameter of about 3 mm to about 26 mm. In some embodiments, an output tubing has an inner diameter of about 3.2 mm to about 25.4 mm. In some embodiments, an output tubing has an inner diameter of about 6.35 mm to about 25.4 mm. In some embodiments, an output tubing has an inner diameter of about 4 mm to about 8 mm. In some embodiments, an output tubing has an inner diameter of about 20 mm to about 30 mm.

### Method of Transfecting Host Cells Using an In-Line Complexer

The present disclosure, among other things, provides methods for transfection of a host cell comprising: combining nucleic acids with a transfection reagent in an in-line complexer to form complexes that comprise nucleic acids and the transfection reagent. The method further comprises introducing the complexes into a vessel that comprises host cells under conditions that lead to transfection of the host cells with the nucleic acids.

In some embodiments, an in-line complexer used in a method disclosed herein comprises (a) a first input tubing in communication at a proximal end to a source that comprises the nucleic acids, (b) a second input tubing in communication at a proximal end to a source that comprises the transfection reagent, and (c) an output tubing that is in communication: (i) at a proximal end to a distal end of the first input tubing and a distal end of the second input tubing; and (ii) at a distal end to the vessel that comprises the host cells.

In some embodiments, nucleic acids used in a method disclosed herein comprise one or more vectors. In some embodiments, nucleic acids disclosed herein comprise one or more vectors encoding: (i) at least one payload flanked by an AAV inverted terminal repeat (ITR) on either side of the at least one payload, (ii) at least one AAV Rep polypeptide, (iii) at least one AAV Cap polypeptide, and/or (iv) at least one Adenoviral helper polypeptide.

A host cell (e.g., a mammalian host cell, e.g., a HEK293) can be transfected with: at least one helper polypeptide (e.g., at least one Ad2 helper polypeptides), at least one Rep polypeptide or a fragment thereof, at least one Cap polypeptide or a fragment thereof, and at least one payload (e.g., for polypeptide expression or an inhibitory or guide nucleic acid).

In some embodiments, a transfection method disclosed herein is or comprises transient transfection. In some embodiments, a transient transfection method is a suspension transient transfection (sTT). In some embodiments, a transient transfection method is an adherent transient transfection.

In some embodiments, the disclosure provides transfected host cells comprising two, three, or four vectors as described herein.

In some embodiments, the method comprises transfecting a host cell with three vectors. In some embodiments, the three vectors comprise: (i) a first vector encoding at least one payload flanked by an AAV ITR on either side of the at least one payload, (ii) a second vector encoding at least one AAV Rep polypeptide and at least one AAV Cap polypeptide, and (iii) a third vector encoding at least one Adenoviral helper polypeptide.

In some embodiments, the method comprises transfecting a host cell with two vectors. In some embodiments, the two vectors comprises (i) a first vector encoding at least one AAV Cap polypeptide and at least one payload flanked by an AAV ITR on either side of the at least one payload; and (ii) a second vector encoding at least one Adenoviral helper polypeptide and at least one AAV Rep polypeptide.

Transfection methods disclosed herein comprise transfection of nucleic acids (e.g., comprising one or more vector) with any transfection reagent known to a skilled person for introducing nucleic acid molecules into host cells (e.g., mammalian cells, such as HEK293). In some embodiments, a transfection reagent comprises a lipid, a polymer, or a combination thereof. In some embodiments, a transfection reagent is a reagent that can form a complex with the nucleic acids.

In some embodiments, a transfection reagent comprise a polymer, a lipid, or both a polymer and a lipid. In some embodiments, a transfection reagent is or comprises a polymer. In some embodiments, a transfection reagent is or comprises lipid. In some embodiments, a transfection reagent comprises a polymer and a lipid.

In some embodiments, a transfection reagent is or comprises a polymer, e.g., a cationic polymer. In some embodiments, a transfection reagent comprises polyethyleneimine (PEI), FectoVIR, TransIT-VirusGEN, or a combination thereof. In some embodiments, a transfection reagent is or comprises polyethyleneimine (PEI).

In some embodiments, host cells are transfected with PEI. In some embodiments, host cells are transfected with a weight (wt.) ratio of DNA to transfection reagent (e.g., PEI) of about 1:1 to about 1:2, about 1:1 to about 1:5, or about 1:1 to about 1:10, e.g., about 1:0.05, about 1:1, about 1:1.25, about 1:1.5, about 1:2, about 1:2.5, about 1:3, about 1:3.5, about 1:4, about 1:4.5, about 1:5, about 1:6, about 1:7, about 1:8, about 1:9, or about 1:10. In some embodiments, a wt. ratio of DNA to transfection reagent is dependent on cell culture density (e.g., of adherent or suspension host cells).

In some embodiments, a vector mass ratio of: (i) a first vector encoding at last one payload to (ii) a second vector encoding at least one Rep polypeptide and/or at least one Cap polypeptide to (iii) a third vector encoding at least one helper polypeptide is used in a method of transfection disclosed herein. In some embodiments, a vector mass ratio of: (i) a first vector encoding at last one payload to (ii) a second vector encoding at least one Rep polypeptide and/or at least one Cap polypeptide to (iii) a third vector encoding at least one helper polypeptide is about 1:1:1. In some embodiments, a vector mass ratio of: (i) a first vector encoding at last one payload to (ii) a second vector encoding at least one Rep polypeptide and/or at least one Cap polypeptide to (iii) a third vector encoding at least one helper polypeptide is not about 1:1:1.

In some embodiments, a vector mass ratio of: (i) a first vector encoding at last one payload to (ii) a second vector encoding at least one Rep polypeptide and/or at least one Cap polypeptide to (iii) a third vector encoding at least one helper polypeptide is about 1:0.5:1, about 1:1:2, about 1:1:3, about 1:1:4, about 1:1:5, about 1:1:6, about 1:1:7, about 1:1:8, about 1:1:9, about 1:1:10, about 5:10:1, about 1:0.5:2, about 1:0.5:10, about 1:0.5:5, about 0.5:5:1, about 1:10:20, about 1:2:1, about 1:3:1, about 1:4:1, about 1:5:1, about 1:6:1, about 1:7:1, about 1:8:1, about 1:9:1, about 1:10:1, about 10:1:1, about 9:1:1, about 8:1:1, about 7:1:1, about 6:1:1, about 6:1:1, about 4:1:1, about 3:1:1, about 2:1:1, or about 1:0.5:5.

In some embodiments, a vector mass ratio of: (i) a first vector encoding at last one payload to (ii) a second vector encoding at least one Rep polypeptide and/or at least one Cap polypeptide to (iii) a third vector encoding at least one helper polypeptide is about 1:0.5:1 to about 1:0.5:10; about 1:1:1 to about 1:1:10; about 0.5:1:1 to about 5:1:1; about 1:1:1 to about 1:10:1; or about 1:1:1 to about 10:1:1.

### Nucleic Acid and Transfection Reagent Volumes

According to any of the methods disclosed herein, a composition comprising nucleic acids is introduced into a first tubing of an in-line complexer and a composition comprising a transfection reagent is introduced into a second tubing. The amount of a composition introduced in a first tubing and/or a second tubing of an in-line complexer can vary depending, e.g., on the vessel used in the transfection method.

In some embodiments, a composition comprising a transfection reagent in an amount of about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.8%, about 0.1% to about 0.6%, about 0.1% to about 0.4%, about 0.1% to about 0.2%, about 0.1% to about 10%, about 0.2% to about 10%, about 0.4% to about 10%, about 0.6%, to about 10%, about 0.8% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 4% to about 9%, or about 5% to about 8% of the volume of a vessel used in a transfection method is introduced into a first tubing.

In some embodiments, a composition comprising nucleic acids in an amount of about 0.1%, about 0.2%, about 0.4%, about 0.6%, about 0.8%, about 1%, about 2%, about 3%, about 4%, about 5%, about 6%, about 7%, about 8%, about 9% or about 10% of the volume of a vessel used in a transfection method is introduced into a first tubing. In some embodiments, a composition comprising nucleic acids in an amount of about 5% of a vessel volume is introduced into a first tubing.

In some embodiments, about 100 mL, about 150 mL, about 200 mL, about 250 mL, about 300 mL, about 400 mL, about 500 mL, about 600 mL, about 700 mL, about 800 mL, about 900 mL, about 1000 mL, about 2000 mL, about 3000 mL, about 4000 mL, about 5000 mL, about 6000 mL, about 7000 mL, about 8000 mL, about 9000 mL, about 10,000 mL, about 20,000 mL, about 30,000 mL, about 40,000 mL, about 50,000 mL, about 60,000 mL, about 70,000 mL, about 80,000 mL, about 90,000 mL, about 100,000 mL, about 110,000 mL, about 120,000 mL, about 130,000 mL, about 140,000 mL, about 150,000 mL of a composition comprising nucleic acids is introduced into a first tubing.

In some embodiments, about 100 mL to about 150,000 mL, about 150 mL to about 150,000 mL, about 200 mL to about 150,000 mL, about 250 mL to about 150,000 mL, about 300 mL to about 150,000 mL, about 400 mL to about 150,000 mL, about 500 mL to about 150,000 mL, about 600 mL to about 150,000 mL, about 700 mL to about 150,000 mL, about 800 mL to about 150,000 mL, about 900 mL to about 150,000 mL, about 1000 mL to about 150,000 mL, about 2000 mL to about 150,000 mL, about 3000 mL to about 150,000 mL, about 4000 mL to about 150,000 mL, about 5000 mL to about 150,000 mL, about 6000 mL to about 150,000 mL, about 7000 mL to about 150,000 mL, about 8000 mL to about 150,000 mL, about 9000 mL to about 150,000 mL, about 10,000 mL to about 150,000 mL, about 20,000 mL to about 150,000 mL, about 30,000 mL to about 150,000 mL, about 40,000 mL to about 150,000 mL, about 50,000 mL to about 150,000 mL, about 60,000 mL to about 150,000 mL, about 70,000 mL to about 150,000 mL, about 80,000 mL to about 150,000 mL, about 90,000 mL to about 150,000 mL, about 100,000 mL to about 150,000 mL, about 110,000 mL to about 150,000 mL, about 120,000 mL to about 150,000 mL, about 130,000 mL to about 150,000 mL, about 140.00 mL to about 150,000 mL of a composition comprising nucleic acids is introduced into a first tubing.

In some embodiments, about 100 mL to about 140,000 mL, about 100 mL to about 130,000 mL, about 100 mL to about 120,000 mL, about 100 mL to about 110,000 mL, about 100 mL to about 100.00 mL, about 100 mL to about 90,000 mL, about 100 mL to about 80,000 mL, about 100 mL to about 70,000 mL, about 100 mL to about 60,000 mL, about 100 mL to about 50,000 mL, about 100 mL to about 40,000 mL, about 100 mL to about 30,000 mL, about 100 mL to about 20,000 mL, about 100 mL to about 10,000 mL, about 100 mL to about 9000 mL, about 100 mL to about 8000 mL, about 100 mL to about 7000 mL, about 100 mL to about 6000 mL, about 100 mL to about 5000 mL, about 100 mL to about 4000 mL, about 100 mL to about 3000 mL, about 100 mL to about 2000 mL, about 100 mL to about 1000 mL, about 100 mL to about 900 mL, about 100 mL to about 800 mL, about 100 mL to about 700 mL, about 100 mL to about 600 mL, about 100 mL to about 500 mL, about 100 mL to about 400 mL, about 100 mL to about 300 mL, about 100 mL to about 200 mL, about 100 mL to about 150 ml of a composition comprising nucleic acids is introduced into a first tubing.

In some embodiments, about 150 mL to about 100,000 mL of a composition comprising nucleic acids is introduced into a first tubing.

In some embodiments, a composition comprising a transfection reagent in an amount of about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.8%, about 0.1% to about 0.6%, about 0.1% to about 0.4%, about 0.1% to about 0.2%, about 0.1% to about 10%, about 0.2% to about 10%, about 0.4% to about 10%, about 0.6%, to about 10%, about 0.8% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 4% to about 9%, or about 5% to about 8% of the volume of a vessel used in a transfection method is introduced into a second tubing.

In some embodiments, a composition comprising a transfection reagent in an amount of about 0.1%, about 0.2%, about 0.4%, about 0.6%, about 0.8%, about 1%, about 2%, about 3%, about 4%, about 5%, about 6%, about 7%, about 8%, about 9% or about 10% of the volume of a vessel used in a transfection method is introduced into a second tubing. In some embodiments, a composition comprising a transfection reagent in an amount of about 5% of a vessel volume is introduced into a second tubing.

In some embodiments, about 100 mL, about 150 mL, about 200 mL, about 250 mL, about 300 mL, about 400 mL, about 500 mL, about 600 mL, about 700 mL, about 800 mL, about 900 mL, about 1000 mL, about 2000 mL, about 3000 mL, about 4000 mL, about 5000 mL, about 6000 mL, about 7000 mL, about 8000 mL, about 9000 mL, about 10,000 mL, about 20,000 mL, about 30,000 mL, about 40,000 mL, about 50,000 mL, about 60,000 mL, about 70,000 mL, about 80,000 mL, about 90,000 mL, about 100,000 mL, about 110,000 mL, about 120,000 mL, about 130,000 mL, about 140,000 mL, about 150,000 mL of a composition comprising a transfection reagent is introduced into a second tubing.

In some embodiments, about 100 mL to about 150,000 mL, about 150 mL to about 150,000 mL, about 200 mL to about 150,000 mL, about 250 mL to about 150,000 mL, about 300 mL to about 150,000 mL, about 400 mL to about 150,000 mL, about 500 mL to about 150,000 mL, about 600 mL to about 150,000 mL, about 700 mL to about 150,000 mL, about 800 mL to about 150,000 mL, about 900 mL to about 150,000 mL, about 1000 mL to about 150,000 mL, about 2000 mL to about 150,000 mL, about 3000 mL to about 150,000 mL, about 4000 mL to about 150,000 mL, about 5000 mL to about 150,000 mL, about 6000 mL to about 150,000 mL, about 7000 mL to about 150,000 mL, about 8000 mL to about 150,000 mL, about 9000 mL to about 150,000 mL, about 10,000 mL to about 150,000 mL, about 20,000 mL to about 150,000 mL, about 30,000 mL to about 150,000 mL, about 40,000 mL to about 150,000 mL, about 50,000 mL to about 150,000 mL, about 60,000 mL to about 150,000 mL, about 70,000 mL to about 150,000 mL, about 80,000 mL to about 150,000 mL, about 90,000 mL to about 150,000 mL, about 100,000 mL to about 150,000 mL, about 110,000 mL to about 150,000 mL, about 120,000 mL to about 150,000 mL, about 130,000 mL to about 150,000 mL, about 140.00 mL to about 150,000 mL of a composition comprising a transfection reagent is introduced into a second tubing.

In some embodiments, about 100 mL to about 140,000 mL, about 100 mL to about 130,000 mL, about 100 mL to about 120,000 mL, about 100 mL to about 110,000 mL, about 100 mL to about 100.00 mL, about 100 mL to about 90,000 mL, about 100 mL to about 80,000 mL, about 100 mL to about 70,000 mL, about 100 mL to about 60,000 mL, about 100 mL to about 50,000 mL, about 100 mL to about 40,000 mL, about 100 mL to about 30,000 mL, about 100 mL to about 20,000 mL, about 100 mL to about 10,000 mL, about 100 mL to about 9000 mL, about 100 mL to about 8000 mL, about 100 mL to about 7000 mL, about 100 mL to about 6000 mL, about 100 mL to about 5000 mL, about 100 mL to about 4000 mL, about 100 mL to about 3000 mL, about 100 mL to about 2000 mL, about 100 mL to about 1000 mL, about 100 mL to about 900 mL, about 100 mL to about 800 mL, about 100 mL to about 700 mL, about 100 mL to about 600 mL, about 100 mL to about 500 mL, about 100 mL to about 400 mL, about 100 mL to about 300 mL, about 100 mL to about 200 mL, about 100 mL to about 150 ml of a composition comprising a transfection reagent is introduced into a second tubing.

In some embodiments, about 150 mL to about 100,000 mL of a composition comprising a transfection reagent is introduced into a second tubing.

In some embodiments, nucleic acids are diluted, e.g., in an appropriate dilution media, prior to introducing into a tubing, e.g., a first tubing. In some embodiments, nucleic acids are not diluted, e.g., in an appropriate media, prior to introducing into a tubing, e.g., a first tubing.

In some embodiments, a transfection reagent is diluted, e.g., in an appropriate dilution media, prior to introducing into a tubing, e.g., a second tubing. In some embodiments, a transfection reagent is not diluted, e.g., in an appropriate dilution media, prior to introducing into a tubing, e.g., a second tubing.

In some embodiments, a ratio of the volume of nucleic acids (e.g., diluted nucleic acids) to transfection reagent is about 1:1, about 1:10, about 1:20, about 1:50, about 1:100, about 1:500, about 1:1000, about 10:1, about 20:1, about 50:1, about 100:1, about 500:1, or about 1000:1.

### Nucleic Acid and Transfection Reagent Complexes

As disclosed herein, methods of transfection comprising combining nucleic acids with a transfection reagent in an in-line complexer can lead to the formation of complexes that comprise the nucleic acids and the transfection reagent. Such complexes, e.g., nanoparticles, aid in the delivery of the nucleic acids to host cells.

The stability of complexes comprising nucleic acids and a transfection reagent is time-dependent with peak transfection efficiency at about 5 minutes after the nucleic acid and transfection reagent have been contacted with each other. Increasing the amount of time the nucleic acid and transfection reagent are in contact, increases aggregation of the complexes and reduces transfection efficiency. Accordingly, transfection efficiency of a host cell can be impacted by: (1) the diameter of complexes comprising nucleic acids and a transfection reagent; and (2) the amount of time nucleic acids and transfection reagent are in contact.

In some embodiments, complexes comprising nucleic acids and a transfection reagent have an average diameter of about 100 nm+/−5%, about 150 nm+/−5%, about 200 nm+/−5%, about 250 nm+/−5%, about 300 nm+/−5%, about 350 nm+/−5%, about 400 nm+/−5%, about 450 nm+/−5%, about 500 nm+/−5%, about 550 nm+/−5%, about 600 nm+/−5%, about 650 nm+/−5%, about 700 nm+/−5%, about 800 nm+/−5%, about 900 nm+/−5%, or about 1000 nm+/−5%.

In some embodiments, a diameter of a complex comprising nucleic acids and a transfection reagent is measured with a microscopy method, e.g., using a dynamic light scattering instrument as described in Example 1 herein. Other microscopy methods that can be used to measure the size of particles can be used to measure the diameter of complex comprising nucleic acids and a transfection reagent.

In some embodiments, complexes comprising nucleic acids and a transfection reagent have an average diameter about 100 nm to about 1000 nm, about 100 nm to about 900 nm, about 100 nm to about 800 nm, about 100 nm to about 700 nm, about 100 nm to about 650 nm, about 100 nm to about 600 nm, about 100 nm to about 550 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 1000 nm, about 200 nm to about 1000 nm, about 250 nm to about 1000 nm, about 300 nm to about 1000 nm, about 350 nm to about 1000 nm, about 400 nm to about 1000 nm, about 450 nm to about 1000 nm, about 500 nm to about 1000 nm, about 550 nm to about 1000 nm, about 600 nm to about 1000 nm, about 650 nm to about 1000 nm, about 700 nm to about 1000 nm, about 800 nm to about 1000 nm, or about 900 nm to about 1000 nm.

In some embodiments, complexes comprising nucleic acids and a transfection reagent have an average diameter about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 800 nm, about 900 nm, or about 1000 nm.

In some embodiments, complexes comprising nucleic acids and a transfection reagent have an average diameter less than 700 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm.

In some embodiments, at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to about 1000 nm.

In some embodiments, the amount of time a nucleic acid is in contact with a transfection reagent is also referred to as complex time.

In some embodiments, the amount of time a nucleic acid is in contact with a transfection reagent can be controlled by, e.g., modulating a flow rate of a tubing, modulating a length of a tubing, and/or modulating an inner diameter of a tubing. In some embodiments of a method or system disclosed herein, a nucleic acid and a transfection reagent are in contact for a duration of time a nucleic acid and a transfection reagent travel a length of a tubing. In some embodiments, it takes between about 30 seconds to about 3600 seconds for a nucleic acid, a transfection reagent and/or complexes comprising a nucleic acid and a transfection reagent to travel a length of a tubing, e.g., a output tubing.

In some embodiments, it takes about 30 seconds to about 3600 seconds, about 30 seconds to about 3000 seconds, about 30 seconds to about 2500 seconds, about 30 seconds to about 2000 seconds, about 30 seconds to about 1500 seconds, about 30 seconds to about 1000 seconds, about 30 seconds to about 900 seconds, about 30 seconds to about 800 seconds, about 30 seconds to about 700 seconds, about 30 seconds to about 600 seconds, about 30 seconds to about 550 seconds, about 30 seconds to about 500 seconds, about 30 seconds to about 450 seconds, about 30 seconds to about 400 seconds, about 30 seconds to about 350 seconds, about 30 seconds to about 300 seconds, about 30 seconds to about 250 seconds, about 30 seconds to about 200 seconds, about 30 seconds to about 150 seconds, about 30 seconds to about 120 seconds, about 30 seconds to about 115 seconds, about 30 seconds to about 110 seconds, about 30 seconds to about 105 seconds, about 30 seconds to about 100 seconds, about 30 seconds to about 95 seconds, about 30 seconds to about 90 seconds, about 30 seconds to about 85 seconds, about 30 seconds to about 80 seconds, about 30 seconds to about 75 seconds, about 30 seconds to about 70 seconds, about 30 seconds to about 65 seconds, about 30 seconds to about 60 seconds, about 30 seconds to about 55 seconds, about 30 seconds to about 50 seconds, about 30 seconds to about 45 seconds, about 30 seconds to about 40 seconds, or about 30 seconds to about 35 seconds for nucleic acids, a transfection reagent and/or complexes comprising nucleic acids and a transfection reagent to travel the length of a tubing, e.g., a output tubing.

In some embodiments, it takes about 30 seconds to about 3600 seconds, about 35 seconds to about 3600 seconds, about 40 to about 3600 seconds, about 45 to about 3600 seconds, about 50 to about 3600 seconds, about 55 to about 3600 seconds, about 60 to about 3600 seconds, about 65 to about 3600 seconds, about 70 to about 3600 seconds, about 75 to about 3600 seconds, about 80 to about 3600 seconds, about 85 to about 3600 seconds, about 90 to about 3600 seconds, about 95 to about 3600 seconds, about 100 to about 3600 seconds, about 105 to about 3600 seconds, about 110 to about 3600 seconds, about 115 to about 3600 seconds, about 120 to about 3600 seconds, about 150 to about 3600 seconds, about 200 to about 3600 seconds, about 250 to about 3600 seconds, about 300 to about 3600 seconds, about 350 to about 3600 seconds, about 400 to about 3600 seconds, about 450 to about 3600 seconds, about 500 to about 3600 seconds, about 550 seconds to about 3600 seconds, 600 to about 3600 seconds, about 700 to about 3600 seconds, about 800 to about 3600 seconds, about 900 to about 3600 seconds, about 1000 to about 3600 seconds, about 1500 to about 3600 seconds, about 2000 to about 3600 seconds, about 2500 to about 3600 seconds, about 3000 to about 3600 seconds for nucleic acids, a transfection reagent and/or complexes comprising nucleic acids and a transfection reagent to travel the length of a tubing, e.g., a output tubing.

In some embodiments, it takes about 30 seconds, about 35 seconds, about 40 seconds, about 45 seconds, about 50 seconds, about 55 seconds, about 60 seconds, about 65 seconds, about 70 seconds, about 80 seconds, about 85 seconds, about 90 seconds, about 95 seconds, about 100 seconds, about 105 seconds, about 110 seconds, about 115 seconds, about 120 seconds, about 150 seconds, about 200 seconds, about 250 seconds, about 300 seconds, about 350 seconds, about 400 seconds, about 450 seconds, about 500 seconds, about 550 seconds, about 600 seconds, about 700 seconds, about 800 seconds, about 900 seconds, about 1000 seconds, about 1500 seconds, about 2000 seconds, about 2500 seconds, about 3000 seconds or about 3600 seconds for nucleic acids, a transfection reagent and/or complexes comprising nucleic acids and a transfection reagent to travel the length of a tubing, e.g., a output tubing.

In some embodiments, it takes 150 seconds+/−10% for nucleic acids, a transfection reagent and/or complexes comprising nucleic acids and a transfection reagent to travel the length of a tubing, e.g., a output tubing.

In some embodiments, it takes 150 seconds+/−10% for complexes comprising nucleic acids and a transfection reagent to travel the length of a tubing, e.g., a output tubing.

In some embodiments, it takes 150 seconds for nucleic acids, a transfection reagent and/or complexes comprising nucleic acids and a transfection reagent to travel the length of a tubing, e.g., a third output tubing.

In some embodiments, it takes 150 seconds for complexes comprising nucleic acids and a transfection reagent to travel the length of a tubing, e.g., a output tubing.

In some embodiments, transfection complexes are formed between a transfection reagent (e.g., PEI) and one, two, or three of: (i) a first vector encoding at least one Adenovirus 2 (Ad2) helper polypeptides; (ii) a second vector encoding at least one Rep polypeptide and/or at least one Cap polypeptide; and (iii) a third vector encoding at least one payload. In some embodiments, transfection complexes are formed between a transfection reagent and one or two of: (i) a first vector encoding at least one Ad2 helper polypeptides and at least one Rep polypeptide, and (ii) a second vector encoding at least one Cap polypeptide and at least one payload. In some embodiments, transfection complexes are formed for less than 10 minutes+/−15%, e.g., for 9 minutes+/−15%, 8 minutes+/−15%, 7 minutes+/−15%, 6 minutes+/−15%, 5 minutes+/−15%, 4 minutes+/−15%, 3 minutes+/−15%, 2 minutes+/−15%, 1 minute+/−15%, or less.

In some embodiments, complexes comprising nucleic acids and a transfection reagent, e.g., which complexes are formed in an output tubing, are added to a culture vessel. In some embodiments, a culture vessel comprises a bioreactor as described herein.

In some embodiments, complexes comprising nucleic acids and a transfection reagent are added to a culture vessel over a period of time. In some embodiments, the period of time is about 2 minutes to about 150 minutes, about 2 minutes to about 140 minutes, about 2 minutes to about 130 minutes, about 2 minutes to about 120 minutes, about 2 minutes to about 110 minutes, about 2 minutes to about 100 minutes, about 2 minutes to about 90 minutes, about 2 minutes to about 80 minutes, about 2 minutes to about 70 minutes, about 2 minutes to about 60 minutes, about 2 minutes to about 50 minutes, about 2 minutes to about 40 minutes, about 2 minutes to about 30 minutes, about 2 minutes to about 20 minutes, about 2 minutes to about 10 minutes, about 5 minutes to about 150 minutes, about 10 minutes to about 150 minutes, about 15 minutes to about 150 minutes, about 20 minutes to about 150 minutes, about 30 minutes to about 150 minutes, about 40 minutes to about 150 minutes, about 50 minutes to about 150 minutes, about 60 minutes to about 150 minutes, about 70 minutes to about 150 minutes, about 80 minutes to about 150 minutes, about 90 minutes to about 150 minutes, about 100 minutes to about 150 minutes, about 110 minutes to about 150 minutes, about 120 minutes to about 150 minutes, about 130 minutes to about 150 minutes or about 140 minutes to about 150 minutes.

In some embodiments, the period of time is about 2 minutes, about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes or about 150 minutes.

### System for Transfecting Host Cells

In addition to methods of transfecting host cells, the present disclosure also provides systems for transfecting host cells. Such systems comprise an in-line complexer, e.g., as described herein.

In some embodiments, disclosed herein are systems for transfecting host cells with nucleic acids, the system comprising an in-line complexer and a vessel comprising host cells, wherein the in-line complexer comprises (a) a first input tubing in communication at a proximal end to a source that comprises nucleic acids, (b) a second input tubing in communication at a proximal end to a source that comprises a transfection reagent, and (c) an output tubing that is in communication (i) at a proximal end to a distal end of the first input tubing and a distal end of the second input tubing and (ii) at a distal end to the vessel that comprises host cells. In some embodiments, the first input tubing and the second input tubing are each in communication with a pump that has a flow rate of about 1 mL/min to about 5000 mL/min (or any of the subranges disclosed above for methods of transfecting host cells). In some embodiments, the output tubing is about 60 mm to 100,000 mm in length and/or about 0.3 mm to 250 mm in inner diameter (or any of the length and/or inner diameter subranges disclosed above for methods of transfecting host cells).

In some embodiments, a system disclosed herein is used in a method for transfecting host cells with nucleic acids, said method comprising combining nucleic acids with a transfection reagent to form complexes that comprise the nucleic acids and the transfection reagent.

### Host Cells

The present disclosure, among other things, provides host cells for transfection with at least one vector as described herein for production of rAAV particles. A host cell includes a progeny cell of an original cell transfected with at least one vector described herein. A progeny cell of a parental cell may not be substantially identical in morphology or genomic content as a parent cell due to natural, accidental, or deliberate mutation.

Components for a host cell to produce rAAV particles may be provided in trans on at least one vector. A stable host cell may comprise at least one polypeptide to produce rAAV particles using methods known to those of skill in the art. In some embodiments, a stable host cell comprises at least one polypeptide under control of an inducible promoter. In other embodiments, a stable host cell comprises at least one polypeptide under control of a constitutive promoter. For example, a stable host cell (e.g., a HEK293 cell) may comprise a nucleic acid encoding an E1 helper polypeptide under the control of a constitutive promoter. Other stable host cells may be generated by one of skill in the art using routine methods.

Exemplary host cells include prokaryotes or eukaryotes (single-cell or multiple-cell), bacterial cells (e.g., strains of E. coli, Bacillus spp., Streptomyces spp), mycobacteria cells, fungal cells, yeast cells (e.g., S. cerevisiae, S. pombe, P. pastoris, P. methanolica), plant cells, insect cells (e.g., SF-9, SF-21, baculovirus-infected insect cells, or Trichoplusia ni), non-human animal cells, human cells, or cell fusions, such as hybridomas or quadromas. In some embodiments the host cell is a mammalian cell. In some embodiments, the host cell is a human, monkey, ape, hamster, rat, or mouse cell.

In some embodiments, the host cell is selected from a kidney cell (e.g., HEK293, 293 EBNA, MSR 293, MDCK, HaK, or BHK), CHO cell (e.g., CHO K1, DXB-1 1 CHO, or Veggie-CHO), HeLa cell, COS cell (e.g., COS-7), retinal cell, Vero cell, CV1 cell, HepG2 cell, WI38 cell, MRC 5 cell, Colo205 cell, HB 8065 cell, HL-60 cell (e.g., BHK21), Jurkat cell, Daudi cell, A431 cell (epidermal), CV-1 cell, U937 cell, 3T3 cell, L cell, C127 cell, SP2/0 cell, NS-0 cell, MMT 060562 cell, Sertoli cell, BRL 3 A cell, HT1080 cell, myeloma cell, tumor cell, or a cell line derived from an aforementioned cell.

In some embodiments, the host cell comprises a kidney cell (e.g., HEK293, 293 EBNA, MSR 293, MDCK, HaK, or BHK). In certain embodiments, the host cell comprises a HEK293 cell. In some embodiments, the host cell (e.g., a HEK 293 cell) comprises or expresses an E1 polypeptide. In some embodiments, the host cell does not comprise or express an E1 polypeptide. In some embodiments, the host cell comprises a CHO cell (e.g., CHO-K, DXB-1 1 CHO, or Veggie-CHO). In certain embodiments, the host cell comprises a CHO-K cell. In certain embodiments, the host cell comprises a HeLa cell.

In some embodiments, host cells are or comprise suspension cells. In some embodiments, at least 10%+/−15%, at least 15+/−15%, at least 20+/−15%, at least 25+/−15%, at least 30+/−15%, at least 35+/−15%, at least 40+/−15%, at least 45+/−15%, at least 50+/−15%, at least 55+/−15%, at least 60+/−15%, at least 65+/−15%, at least 70+/−15%, at least 75+/−15%, at least 80+/−15%, at least 85+/−15%, at least 90+/−15%, at least 95+/−15%, at least 99+/−15%, or more host cells in culture are suspended.

In some embodiments, prior to transfection, host cells (e.g., adherent or suspended host cells) are seeded at a certain density. In some embodiments, prior to transfection, host cells (e.g., adherent host cells) are seeded at a density of at least about 1.0×10⁴ viable cells (vc)/cm², e.g., at a density of about 1.0×10⁴ vc/cm² to about 2.0×10⁴ vc/cm², e.g., about 1.0×10⁴ vc/cm², about 1.1×10⁴ vc/cm², about 1.2×10⁴ vc/cm², about 1.3×10⁴ vc/cm², about 1.4×10⁴ vc/cm², about 1.5×10⁴ vc/cm², about 1.6×10⁴ vc/cm², about 1.7×10⁴ vc/cm², about 1.8×10⁴ vc/cm², about 1.9×10⁴ vc/cm², or about 2.0×10⁴ vc/cm². In some embodiments, prior to transfection, host cells (e.g., suspended host cells) are seeded at a density of at least 1.0×10⁶ vc/cm²+/−15%, e.g., at a density of 1.0×10⁶ vc/cm²+/−15% to 2.0×10⁶ vc/cm²+/−15%, e.g., 1.0×10⁶ vc/cm²+/−15%, 1.1×10⁶ vc/cm²+/−15%, 1.2×10⁶ vc/cm²+/−15%, 1.3×10⁶ vc/cm²+/−15%, 1.4×10⁶ vc/cm²+/−15%, 1.5×10⁶ vc/cm²+/−15%, 1.6×10⁶ vc/cm²+/−15%, 1.7×10⁶ vc/cm²+/−15%, 1.8×10⁶ vc/cm²+/−15%, 1.9×10⁶ vc/cm²+/−15%, or 2.0×10⁶ vc/cm²+/−15%.

### Vectors

Many forms of vectors can be used in methods of producing rAAV particles described herein. Non-limiting examples of vectors include plasmids, bacteriophage vectors, cosmids, phagemids, artificial chromosomes, and viral vectors (e.g., vectors suitable for gene therapy). A vector genetic element may be delivered by any suitable method known in the art, e.g., to those with skill in nucleic acid manipulation and include genetic engineering, recombinant engineering, and synthetic techniques (See, e.g., Sambrook et al., Molecular Cloning: A Laboratory Manual, Cold Spring Harbor Press, Cold Spring Harbor, N.Y.).

In some embodiments, a vector encodes at least one helper polypeptide. In some embodiments, a vector encodes at least one Rep polypeptide and/or at least one Cap polypeptide. In some embodiments, a vector encodes at least one payload (e.g., for expression of polypeptide or as an inhibitory or guide nucleic acid). In some embodiments, a vector encodes at least one helper polypeptide and at least one Rep polypeptide. In some embodiments, a vector encodes at least one Cap polypeptide and at least one payload.

A vector can include conventional control elements operably linked to a nucleic acid encoding any polypeptide or payload described herein, in a manner that permits transcription, translation and/or expression in a cell transfected with a vector described herein. Expression control sequences include appropriate transcription initiation, termination, promoter and enhancer sequences; efficient RNA processing signals, such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters that are native, constitutive, inducible, and/or tissue-specific, are known in the art and may be included in a vector described herein.

Examples of constitutive promoters include, but are not limited to, a retroviral Rous sarcoma virus (RSV) LTR promoter (optionally with the RSV enhancer), a cytomegalovirus (CMV) promoter (optionally with CMV enhancer), an SV40 promoter, and an dihydrofolate reductase promoter.

Inducible promoters allow regulation of gene expression and can be regulated by exogenously supplied compounds, environmental factors, such as temperature, or the presence of a specific physiological state (e.g., acute phase, a particular differentiation state of the cell, or in replicating cells only). Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system, an ecdysone insect promoter, a tetracycline-repressible system, a tetracycline-inducible system, a RU486-inducible system, and an rapamycin-inducible system. Still other types of inducible promoters that may be useful are regulated by a specific physiological state, such as temperature, acute phase, a particular differentiation state of the cell, or in replicating cells only.

In another embodiment, a native promoter or fragment thereof for a nucleic acid encoding any polypeptide or payload described herein may be used. In some embodiments, other native expression control elements, such as enhancer elements, polyadenylation sites, or Kozak consensus sequences, may also be used to mimic native expression.

### Vector Encoding Helper Polypeptides

The present disclosure, among other things, provides vectors (e.g., plasmids) encoding at least one helper polypeptide. AAV is a helper-dependent DNA parvovirus, which belongs to the genus Dependovirus. Production of recombination AAV requires co-infection with a related virus (e.g., adenovirus, herpes, or vaccinia virus) or a helper vector encoding helper polypeptides, such as structural proteins and proteins for viral genome replication.

A helper vector can comprise nucleotide sequences for non-AAV derived viral and/or cellular functions upon which AAV is dependent for replication, which may include, but are not limited to, activation of gene transcription, stage specific mRNA splicing, DNA replication, synthesis of at least one Cap polypeptide, and/or capsid assembly. Viral-based helper polypeptides can be derived from any known helper viruses such as adenovirus, herpesvirus, vaccinia virus, or a combination thereof. Thus, a helper vector (e.g., a plasmid) for culturing of the host cell can comprise sufficient helper polypeptides to permit packaging of the recombinant AAV vector into the AAV capsid polypeptides.

In some embodiments, a helper vector comprises an Ad2 helper vector. In certain embodiments, a nucleic acid sequence of an Ad2 helper vector is derived from an Adenovirus 2 genome (Genbank Accession No. J01917.1). In some embodiments, a helper vector comprises an Ad5 helper vector. In certain embodiments, a nucleic acid sequence of an Ad5 helper vector is derived from an Adenovirus 5 genome (Genbank Accession No. AY601635).

Helper polypeptides can comprise at least one, two, three, or four of E1, E2A, E4, or VA RNA. In some embodiments, E1 comprises E1a and/or E1b. In some embodiments, one or both of E2A and VA RNA increase stability and/or efficiency of AAV mRNA translation, such as for cap gene transcripts. In some embodiments, E4 facilitates DNA replication. In some embodiments, E1a comprises a transactivator (e.g., regulating activity of at least one Ad gene, AAV rep gene, and/or AAV cap gene). In some embodiments, E1b comprises a viral mRNA transport. Helper polypeptides are described in further detail in Coura and Nardi, A role for adeno-associated viral vectors in gene therapy, Genetics and Molecular Biology, 31(1): 1-11 (2008), which is hereby incorporated by reference in its entirety.

In some embodiments, a helper vector comprises a selection marker. Exemplary selection markers include, but are not limited to, antibiotic resistance genes. In some embodiments, an antibiotic resistance gene is not a gene encoding penicillin. In some embodiments, an antibiotic resistance gene is not a gene encoding a penicillin-derivative. In some embodiments, an antibiotic resistance gene comprises an antibiotic resistance gene chosen from kanamycin, puromycin, neomycin, hygromycin, blasticidin, gentamycin, Gr18, or zeocin. In certain embodiments, an antibiotic resistance gene comprises an antibiotic resistance gene for kanamycin.

In some embodiments, nucleic acids encoding helper polypeptides are oriented in the same direction (e.g., 5′ to 3′) on a helper vector. In some embodiments, nucleic acids encoding helper polypeptides are transcribed in the same direction from a helper vector. In certain embodiments, helper polypeptides comprise VA RNA and E4 oriented in the same direction on a helper vector. In certain embodiments, helper polypeptides comprise E4 and E2A oriented in the same direction on a helper vector. In certain embodiments, helper polypeptides comprise VA RNA, E4, and E2A oriented from 5′ to 3′ in direction on a helper vector. In some embodiments, a helper vector does not comprise a nucleic acid sequence encoding a Fiber protein or a fragment thereof (e.g., does not comprise a nucleic acid sequence of Genbank Accession No. AP_000226.1 or a fragment thereof).

Vector Encoding Rep and/or Cap Polypeptides

The present disclosure, among other things, provides vectors (e.g., plasmids) encoding at least one Rep polypeptide and/or at least one Cap polypeptide. Production of rAAV particles can include culturing of a host cell with at least one Rep polypeptide and at least one Cap polypeptide. Rep proteins (e.g., one, two, three, or four Rep78, Rep68, Rep52, and Rep40) are involved in viral DNA replication, resolution of replicative intermediates, and generation of single-stranded genomes. In some embodiments, a vector comprises a nucleic acid sequence encoding one, two, three, or four of Rep78, Rep68, Rep52, or Rep40, or a variant of any of the foregoing.

In some embodiments, a Rep polypeptide comprises a nucleic acid sequence derived from an AAV2 serotype. For example, a nucleic acid sequence encoding a Rep polypeptide may be derived from the AAV2 genome (as found in Accession No. NC_001401). In some embodiments, a Rep polypeptide comprises an AAV2 Rep polypeptide operably linked to a p5 and/or p19 promotor (as found in Accession No. NC_001401). In some embodiments, a Rep polypeptide comprises an amino acid sequence of YP_680422.1 or a fragment thereof. In some embodiments, a promoter is operably linked to a nucleic acid sequence encoding at least one Rep polypeptide. In certain embodiments, a promoter operably linked to a nucleic acid sequence encoding at least one Rep polypeptide comprises a p5 and/or p19 promoter. In some embodiments, a wildtype promoter of AAV2 or a variant thereof is operably linked to a nucleic acid sequence encoding at least one Rep polypeptide. In some embodiments, a promoter (e.g., a p5 promoter) regulating expression of at least one Rep polypeptide is located in a different location on a vector than a wildtype promoter of AAV2 or a variant thereof. In certain embodiments, a promoter (e.g., a p5 promoter) is located 3′ of a nucleic acid encoding at least one Rep polypeptide. In certain embodiments, a promoter (e.g., a p5 promoter) is located 5′ of a nucleic acid encoding at least one Rep polypeptide.

Cap polypeptides (e.g., VP1, VP2, and VP3) are structural proteins comprising a Capsid. In some embodiments, a vector comprises a nucleic acid sequence encoding one, two, or three of VP1, VP2, and VP3. In some embodiments, a vector comprises a nucleic acid sequence encoding at least one Cap polypeptide and at least one Rep polypeptide. In other embodiments, a vector encodes at least one Cap polypeptide and a separate vector encodes at least one Rep polypeptide.

In some embodiments, a Cap polypeptide comprises a nucleic acid sequence derived from an AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAVl-7/rh.48, AAVl-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-1 1/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu. 1, AAVH2, AAVH-5/hu.3, AAVH6, AAVhEl.1, AAVhER1.14, AAVhErl.16, AAVhErl 0.18, AAVhER1.23, AAVhErl 0.35, AAVhErl 0.36, AAVhErl 0.5, AAVhErl 0.7, AAVhErl 0.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu68, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV-PHP.B, AAV-PHP.N, AAV-PHP.S, AAVrh74, AAV-HSC 1-17, AAV-CBr, AAV-CLv, AAV-CLg, AAV.CAP-B1 to AAV.CAP-B25, serotype, or a variant of any of the foregoing, such as Rec2 or Rec3.

For example, a nucleic acid sequence encoding a Cap polypeptide may be derived from a known AAV genome sequence including, but not limited to: AAV1 Accession No. NC_002077 or AF063497; AAV2 Accession No. NC_001401; AAV3 Accession No. NC_001729; AAV3B Accession No. NC_001863; AAV4 Accession No. NC_001829; AAV5 Accession No. Y18065 or AF085716; Accession No. AAV6 NC_001862; Avian AAV ATCC VR-865 AY186198, AY629583, or NC_004828; Avian AAV strain DA-1 NC_006263, AY629583; or Bovine AAV NC_005889, AY388617. In certain embodiments, a nucleic acid sequence encoding a Cap polypeptide is derived from an AAV genome sequence or a variant thereof as described in U.S. Pat. No. 7,906,111, which is hereby incorporated by reference in its entirety. In certain embodiments, a nucleic acid sequence encoding a Cap polypeptide is derived from an AAV genome sequence or a variant thereof as described in International Publication No. WO 2018/160582, which is hereby incorporated by reference in its entirety.

In certain embodiments, a Cap polypeptide comprises a nucleic acid sequence derived from an AAV2 serotype, or a variant thereof. In certain embodiments, a Cap polypeptide comprises a nucleic acid sequence derived from an AAV2 serotype, or a variant thereof. In certain embodiments, a Cap polypeptide comprises a nucleic acid sequence derived from an AAV5 serotype, or a variant thereof. In certain embodiments, a Cap polypeptide comprises a nucleic acid sequence derived from an AAV8 serotype, or a variant thereof. In certain embodiments, a Cap polypeptide comprises a nucleic acid sequence derived from an AAV9 serotype, or a variant thereof. In certain embodiments, a Cap polypeptide comprises a nucleic acid sequence derived from AAVhu68 or a variant thereof. In certain embodiments, a Cap polypeptide comprises a nucleic acid sequence derived from AAVrh10 or a variant thereof.

In some embodiments, a promoter is operably linked to a nucleic acid sequence encoding at least one Cap polypeptide. In some embodiments, a wildtype promoter of AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAVl-7/rh.48, AAVl-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-1 1/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu. 1, AAVH2, AAVH-5/hu.3, AAVH6, AAVhEl.1, AAVhER1.14, AAVhErl 0.16, AAVhErl 0.18, AAVhER1.23, AAVhErl 0.35, AAVhErl 0.36, AAVhErl 0.5, AAVhErl 0.7, AAVhErl 0.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu68, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV-PHP.B, AAV-PHP.N, AAV-PHP.S, AAVrh74, AAV-HSC 1-17, AAV-CBr, AAV-CLv, AAV-CLg, AAV.CAP-B1 to AAV.CAP-B25, or a variant of any of the foregoing, is operably linked to a nucleic acid sequence encoding at least one Rep polypeptide. In certain embodiments, a p40 promoter is operably linked to a nucleic acid sequence encoding at least one Cap polypeptide.

### Vector Encoding Payload

The present disclosure, among other things, provides vectors (e.g., plasmids) encoding at least one payload. A payload sequence is generally a sequence of interest that is desired to be introduced into a cell, tissue, organ, or organism.

In some embodiments, a payload is flanked by inverted terminal repeats (ITRs). The AAV sequences of a rAAV vector typically comprise cis-acting 5′ and 3′ inverted terminal repeat (ITR) sequences (See, e.g., B. J. Carter, in “Handbook of Parvoviruses,” ed., P. Tijsser, CRC Press, pp. 155-168 (1990), which is hereby incorporated by reference in its entirety). ITR sequences are typically about 145 bp in length. In some embodiments, one or both of a 5′ITR or a 3′ ITR nucleic acid sequence are modified relative to a known ITR nucleic acid sequence. Modification of ITR nucleic acid sequences is within one of skill in the art (See, e.g., Sambrook et al, “Molecular Cloning. A Laboratory Manual”, 2d ed., Cold Spring Harbor Laboratory, New York (1989); and K. Fisher et al., J Virol., 70:520 532 (1996), each of which is hereby incorporated by reference in its entirety). AAV ITR sequences may be obtained from any known AAV, including mammalian AAV types.

In some embodiments, a payload is a heterologous protein with a therapeutic purpose, e.g., an enzyme, cytokine, antibody, receptor, fusion protein, or chimeric polypeptide. In some embodiments, a payload is linked to a secretion signal sequence for secretion of an expressed polypeptide from a host cell. In some embodiments, a payload is a heterologous nucleic acid with a therapeutic purpose, e.g., an miRNA, siRNA, shRNA, mRNA, snRNA, or CRISPR/Cas guide RNA, or a precursor thereof. One of skill in the art will recognize that a payload can be selected from any heterologous protein or nucleic acid of interest. In some embodiments, a payload sequence comprises one or more aptamer-binding domains or polypeptide-binding domains (e.g., transcription factor binding domains). A vector will also typically include other regulatory elements (e.g., promoters, introns, and/or enhancers) to regulate expression or amount of a payload in a cell or tissue.

In accordance with various embodiments, a payload sequence can be of any length, e.g., between 2 and 10,000 nucleotides in length or any integer value there between. In some embodiments, a nucleic acid sequence encoding a payload comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides. In some embodiments, a nucleic acid sequence encoding a payload comprises between 50 and 25,000 nucleotides in length, between 100 and 20,000 nucleotides in length, between 500 and 10,000 nucleotides in length, between 1,000 and 8,000 nucleotides in length, and/or between 2,000 and 5,000 nucleotides in length.

### Culture Vessels and Culturing Parameters

The present disclosure, among other things, provides methods for culturing of a host cell with at least one vector described herein for production of rAAV particles. A wide variety of growth media (e.g., mammalian growth media) may be used in accordance with the present invention. In certain embodiments, cells may be grown in one of a variety of chemically defined media, wherein the components of the media are both known and controlled. In certain embodiments, cells may be grown in a complex medium, in which not all components of the medium are known and/or controlled.

A culture of host cells can be prepared in any medium suitable for a particular cell type being cultured. In some embodiments, a host cell medium comprises, e.g., inorganic salts, carbohydrates (e.g., sugars, such as glucose, galactose, maltose, or fructose), amino acids, vitamins (e.g., B group vitamins (e.g., B12), vitamin A, vitamin E, riboflavin, thiamine, or biotin), fatty acids (e.g., cholesterol or steroids), proteins (e.g., albumin, transferrin, fibronectin, or fetuin), serum (e.g., albumins, growth factors, or growth inhibitors, such as, fetal bovine serum, newborn calf serum, or horse serum), trace elements (e.g., zinc, copper, selenium, or tricarboxylic acid intermediates), hydrolysates (e.g., derived from plant or animal sources), or combinations thereof.

Commercially available media can be used for culturing host cells described herein. Exemplary media can include, but is not limited to, Dulbecco's Modified Eagle's Medium ([DMEM], Sigma), FreeStyle™ F17 Expression Medium (ThermoFisher), DMEM/F12 medium (Invitrogen), CD OptiCHO™ medium (Invitrogen), CD EfficientFeed™ media (Invitrogen), Cell Boost (HyClone™) media (GE Life Sciences), BalanCD™ CHO Feed (Irvine Scientific), BD Recharge™ (Becton Dickinson), Cellvento Feed™ (EMD Millipore), Ex-cell CHOZN Feed™ (Sigma-Aldrich), CHO Feed Bioreactor Supplement (Sigma-Aldrich), SheffCHO™ (Kerry), Zap-CHO™ (Invitria), ActiCHO™ (PAA/GE Healthcare), Minimal Essential Medium (Sigma), or RPMI-1640 (Sigma). Media can be supplemented as necessary with hormones and/or other growth factors (e.g., insulin, transferrin, or epidermal growth factor), salts (e.g., sodium chloride, calcium, magnesium, or phosphate), buffers (e.g., HEPES), nucleosides (e.g., adenosine or thymidine), antibiotics (e.g., kanamycin, puromycin, neomycin, hygromycin, blasticidin, gentamycin, Gr18, or zeocin), trace elements, lipids (e.g., linoleic or other fatty acids), or glucose or an equivalent energy source. In some embodiments, the media for culturing host cells comprises glutamine or a glutamine dipeptide. In some embodiments, the media for culturing host cells comprises a surfactant. In some embodiments, the nutrient media is serum-free media, a protein-free media, or a chemically defined media. Any other necessary supplements can also be included at appropriate concentrations that would be known to those skilled in the art.

After culturing of host cells as described herein, a plurality of rAAV particles are recovered. In some embodiments, rAAV particles are recovered by lysing host cells and recovering rAAV particles from lysate, e.g., after centrifugation. In some embodiments, rAAV particles are recovered from culture supernatant. In some embodiments, a lysis solution for host cells comprises chemical reagents, e.g., detergents (e.g., sodium dodecyl sulfate (SDS), ethyl trimethyl ammonium bromide, Triton X-100, bile salts, such as cholate, or zwitterionic detergents, such as CHAPS). In some embodiments, a lysis solution for host cells comprises a salt (e.g., NaCl) and a high pH (e.g., a pH of greater than about 7). In some embodiments, rAAV particles are purified using purification methods, such as chromatography (e.g., affinity chromatography or ion-exchange chromatography (e.g., cation exchange chromatography)) or filtration (e.g., UF/DF filtration)).

In some embodiments, a plurality of rAAV particles are produced in a large-scale preparation. In some embodiments, a large-scale preparation of host cells (e.g., suspension host cells) is at least 3 liters+/−15% of culture media, 10 liters+/−15% of culture media, e.g., between 50 liters+/−15% to 1000 liters+/−15% of culture media or between 50 liters+/−15% to 2000 liters+/−15% of culture media, e.g., at least 20 liters+/−15%, 30 liters+/−15%, 40 liters+/−15%, 50 liters+/−15%, 55 liters+/−15%, 60 liters+/−15%, 65 liters+/−15%, 70 liters+/−15%, 75 liters+/−15%, 80 liters+/−15%, 85 liters+/−15%, 90 liters+/−15%, 95 liters+/−15%, 100 liters+/−15%, 200 liters+/−15%, 300 liters+/−15%, 400 liters+/−15%, 500 liters+/−15%, 600 liters+/−15%, 700 liters+/−15%, 800 liters+/−15%, 900 liters+/−15%, 1,000 liters+/−15%, 1,250 liters+/−15%, 1,500 liters+/−15%, 1,750 liters+/−15%, 2,000 liters+/−15%, or more of culture media.

In some embodiments, a large-scale preparation of host cells (e.g., adherent host cells) is at least 5 m²+/−15% of culture media, e.g., between 5 m²+/−15% to 500 m²+/−15% of culture media, e.g., at least 5 m²+/−15%, 10 m²+/−15%, 15 m²+/−15%, 20 m²+/−15%, 25 m²+/−15%, 20 m²+/−15%, 35 m²+/−15%, 40 m²+/−15%, 45 m²+/−15%, 50 m²+/−15%, 55 m²+/−15%, 60 m²+/−15%, 65 m²+/−15%, 75 m²+/−15%, 80 m²+/−15%, 85 m²+/−15%, 90 m²+/−15%, 95 m²+/−15%, 100 m²+/−15%, 150 m²+/−15%, 175 m²+/−15%, 200 m²+/−15%, 225 m²+/−15%, 250 m²+/−15%, 275 m²+/−15%, 300 m²+/−15%, 325 m²+/−15%, 330 m²+/−15%, 340 m²+/−15%, 350 m²+/−15%, 375 m²+/−15%, 400 m²+/−15%, 425 m²+/−15%, 450 m²+/−15%, 475 m²+/−15%, 500 m²+/−15%, 600 m²+/−15%, 700 m²+/−15%, 800 m²+/−15%, 900 m²+/−15%, 1000 m²+/−15%, 1100 m²+/−15%, 1200 m²+/−15%, 1300 m²+/−15%, 1400 m²+/−15%, 1500 m²+/−15%, 1600 m²+/−15%, 1700 m²+/−15%, 1800 m²+/−15%, 1900 m²+/−15%, 2000 m²+/−15%, 2500 m²+/−15%, 3000 m²+/−15%, 3500 m²+/−15%, 4000 m²+/−15%, 4500 m²+/−15%, 5000 m²+/−15%, 5500 m²+/−15%, 6000 m²+/−15%, 6500 m²+/−15%, 7000 m²+/−15%, 7500 m²+/−15%, 8000 m²+/−15%, 8500 m²+/−15%, 9000 m²+/−15%, 9500 m²+/−15%, 10,000 m²+/−15%, 11,000 m²+/−15%, 12,000 m²+/−15%, 13,000 m²+/−15%, 14,000 m²+/−15%, 15,000 m²+/−15%, or more of culture media.

A host cell can be cultured in a cell culture vessel or a bioreactor. In some embodiments, a cell culture vessel is suitable for/used for culturing adherent cells. In other embodiments, a cell culture vessel is suitable for/used for culturing suspension cells. Exemplary cell culture vessels include 35 mm, 60 mm, 100 mm, or 150 mm dishes, multi-well plates (e.g., 6-well, 12-well, 24-well, 48-well, or 96 well plates), or flasks (e.g., T-flasks, e.g., T-25, T-75, or T-160 flasks), or shaker flasks.

In some embodiments, a host cell is cultured in a bioreactor. In some embodiments, a bioreactor is suitable for/used for culturing adherent cells. In some embodiments, a bioreactor is suitable for/used for culturing suspension cells. A bioreactor can be, e.g., a continuous flow batch bioreactor, a perfusion bioreactor, a batch process bioreactor, or a fed batch bioreactor. An exemplary bioreactor is a fixed bed bioreactor, e.g., an iCELLis® bioreactor (used for culturing adherent cells). A bioreactor can be maintained under conditions sufficient to produce rAAV particles. Culture conditions can be modulated to optimize yield, purity, or structure of rAAV particles.

In some embodiments, a bioreactor comprises a plurality of host cells. In some embodiments, host cells in a bioreactor comprise viable cells (vc).

In some embodiments, a bioreactor comprises at least about 1×10⁶, about 1×10⁷, about 1×10⁸, about 1×10⁹, about 1×10¹⁰, about 1×10¹¹, about 1×10¹², about 1×10¹³, or about 1×10¹⁴ host cells (e.g., viable host cells). In some embodiments, a bioreactor comprises between 1×10⁶ to 1×10¹⁴ host cells; between 1×10⁶ to 0.5×10¹⁴ host cells; between 1×10⁶ to 1×10¹³ host cells; between 1×10⁶ to 0.5×10¹³ host cells; between 1×10⁶ to 1×10¹² host cells; between 1×10⁶ to 0.5×10¹² host cells; between 1×10⁶ to 1×10¹¹ host cells; between 1×10⁶ to 0.5×10¹¹ host cells; between 1×10⁶ to 1×10¹⁰ host cells; between 1×10⁶ to 0.5×10¹⁰ host cells; between 1×10⁶ to 1×10⁹ host cells; between 1×10⁶ to 0.5×10⁹ host cells; between 1×10⁶ to 1×10⁸ host cells; between 1×10⁶ to 0.5×10⁸ host cells; between 1×10⁶ to 1×10⁷ host cells; between 1×10⁶ to 0.5×10⁷ host cells; between 0.5×10⁷ to 1×10¹⁴ host cells; between 1×10⁸ to 1×10¹⁴ host cells; between 0.5×10⁹ to 1×10¹⁴ host cells; between 1×10⁹ to 1×10¹⁴ host cells; between 0.5×10¹⁰ to 1×10¹⁴ host cells; between 1×10¹⁰ to 1×10¹⁴ host cells; between 0.5×10¹¹ to 1×10¹⁴ host cells; between 1×10¹¹ to 1×10¹⁴ host cells; between 0.5×10¹² to 1×10¹⁴ host cells; between 1×10¹² to 1×10¹⁴ host cells; between 0.5×10¹³ to 1×10¹⁴ host cells; between 1×10¹³ to 1×10¹⁴ host cells; or between 0.5×10¹³ to 1×10¹⁴ host cells.

In some embodiments, a bioreactor comprises about 0.5 million host cells/mL, about 1 million host cells/mL, about 1.5 million host cells/mL, about 2 million host cells/mL, about 2.5 million host cells/mL, about 3 million host cells/mL, about 3.5 million host cells/mL, about 4 million host cells/mL, about 4.5 million host cells/mL, about 5 million host cells/mL, about 5.5 million host cells/mL, about 6 million host cells/mL, about 7 million host cells/mL, about 8 million host cells/mL, about 9 million host cells/mL, about 10 million host cells/mL. In some embodiments, host cells in a bioreactor comprise viable cells (vc).

In some embodiments, a bioreactor comprises at least about 1 liter, about 2 liters, about 3 liters, about 10 liters, about 20 liters, about 30 liters, about 40 liters, about 50 liters, about 55 liters, about 60 liters, about 65 liters, about 70 liters, about 75 liters, about 80 liters, about 85 liters, about 90 liters, about 95 liters, about 100 liters, about 200 liters, about 300 liters, about 400 liters, about 500 liters, about 600 liters, about 700 liters, about 800 liters, about 900 liters, about 1000 liters, about 1500 liters, about 2000 liters, about 3000 liters, about 4000 liters, about 5000, liters, about 6000, liters, about 7000 liters, about 8000 liters, about 9000 liters, or about 10,000 liters of culture media.

In an embodiment, a bioreactor is maintained under conditions that promote growth of a host cell, e.g., at a temperature (e.g., 37° C.) and gas concentration (e.g., 5%-10% CO₂) that is permissive for growth of the host cell. For example, a bioreactor can perform one or more of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and C02 levels, maintenance of pH level, agitation (e.g., stirring), cleaning, and/or sterilization. Exemplary bioreactor units, may contain multiple reactors within a unit, e.g., a unit can comprise 1, 2, 3, 4, 5, 10, 15, 20, 25, 30, 35, 40, 45, 50, 60, 70, 80, 90, or 100, or more bioreactors. Any suitable bioreactor diameter and/or shape can be used. In some embodiments, suitable reactors can be round, e.g., cylindrical. In some embodiments, suitable reactors can be square, e.g., rectangular.

rAAV Particle Production

The present disclosure, among other things, rAAV particles produced using methods described herein. Generally, rAAV particles produced using methods described herein may be of any AAV serotype. AAV serotypes generally have different tropisms to infect different tissues. In some embodiments, an AAV serotype is selected based on a tropism.

In some embodiments, an AAV particle may comprise or be based on a serotype selected from any of the following serotypes, and variants thereof, including, but not limited to: AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAVl-7/rh.48, AAVl-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-1 1/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu. 1, AAVH2, AAVH-5/hu.3, AAVH6, AAVhEl. 1, AAVhER1.14, AAVhErl 0.16, AAVhErl 0.18, AAVhER1.23, AAVhErl 0.35, AAVhErl 0.36, AAVhErl 0.5, AAVhErl 0.7, AAVhErl 0.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu68, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV-PHP.B, AAV-PHP.N, AAV-PHP.S, AAVrh74, AAV-HSC 1-17, AAV-CBr, AAV-CLv, AAV-CLg, and/or AAV.CAP-B1 to AAV.CAP-B25.

In certain embodiments, an AAV particle comprises an AAV2 serotype or a variant thereof. In certain embodiments, an AAV particle comprises an AAV5 serotype or a variant thereof. In certain embodiments, an AAV particle comprises an AAV8 serotype or a variant thereof. In certain embodiments, an AAV particle comprises an AAV9 serotype or a variant thereof. In certain embodiments, an AAV particle comprises AAVhu68 or a variant thereof. In certain embodiments, an AAV particle comprises AAVrh10 or a variant thereof.

In some embodiment, a plurality of rAAV particles are produced with methods described herein at a higher titer, e.g., such there is improved rAAV particle production. In some embodiments, the improved production comprises a higher yield of the plurality of rAAV particles relative to a plurality of rAAV particles produced with a helper vector comprising a nucleic acid sequence of an antibiotic resistance gene other than KanR (e.g., an Ampicillin resistance gene). In some embodiments, a high titer is relative to AAV particles produced from a reference helper vector (e.g., an Ad5 vector, e.g., an Ad5 vector described herein), e.g., under otherwise identical conditions.

In some embodiments, a high titer is greater than 7.0×10⁹ vg/mL+/−15%, e.g., when cultured in suspension. In some embodiments, a high titer is greater than about 7.0×10⁹ vg/mL, e.g., greater than about 7.5×10⁹ vg/mL, 8.0×10⁹ vg/mL, 8.5×10⁹ vg/mL, 9.0×10⁹ vg/mL, 1.0×10¹⁰ vg/mL, 1.5×10¹⁰ vg/mL, 2.0×10¹⁰ vg/mL, 2.5×10¹⁰ vg/mL, 3.0×10¹⁰ vg/mL, 3.5×10¹⁰ vg/mL, 4.0×10¹⁰ vg/mL, 4.5×10¹⁰ vg/mL, 5.0×10¹⁰ vg/mL, 5.5×10¹⁰ vg/mL, 6.0×10¹⁰ vg/mL, 6.5×10¹⁰ vg/mL, 7.0×10¹⁰ vg/mL, 7.5×10¹⁰ vg/mL, 8.0×10¹⁰ vg/mL, 8.5×10¹⁰ vg/mL, 9.0×10¹⁰ vg/mL, 9.5×10¹⁰ vg/mL, 1.0×10¹¹ vg/mL, 1.5×10¹¹ vg/mL, 2.0×10¹¹ vg/mL, or higher, e.g., when cultured in suspension.

In some embodiments, a high titer of rAAV particles is at least about 7.0×10⁹ vg/cm², about 7.5×10⁹ vg/cm², about 8.0×10⁹ vg/cm², about 8.5×10⁹ vg/cm², about 9.0×10⁹ vg/cm², about 9.5×10⁹ vg/cm², about 1.0×10¹⁰ vg/cm², about 1.5×10¹⁰ vg/cm², or higher, e.g., when cultured in a bioreactor, e.g., a fixed bed bioreactor.

In some embodiments, a high titer of rAAV particles is greater than 5.0×10¹³ vg/m²+/−15%, e.g., greater than 6.0×10¹³ vg/m²+/−15, 7.0×10¹³ vg/m²+/−15, 8.0×10¹³ vg/m²+/−15, 9.0×10¹³ vg/m²+/−15, 1.0×10¹⁴ vg/m²+/−15, 2.0×10¹⁴ vg/m²+/−15, 3.0×10¹⁴ vg/m²+/−15, 4.0×10¹⁴ vg/m²+/−15, 5.0×10¹⁴ vg/m²+/−15, 6.0×10¹⁴ vg/m²+/−15, 7.0×10¹⁴ vg/m²+/−15, 8.0×10¹⁴ vg/m²+/−15, 9.0×10¹⁴ vg/m²+/−15, or more.

In some embodiments, a plurality of rAAV particles described herein is harvested after at least 3 days of culturing. In some embodiments, a plurality of rAAV particles described herein is harvested after at least about 3 days to about 10 days of culturing, e.g., about 3 days to about 7 days, about 3 days to about 5 days, about 4 days to about 9 days, about 4 days to about 8 days, or about 4 days to about 6 days of culturing, e.g., after at least about 3 days, about 4 days, about 5 days, about 6 days, about 7 days, about 8 days, about 9 days, about 10 days, or longer after culturing. In some embodiments, a plurality of rAAV particles produced with methods described herein is substantially free of one or both of a helper adenovirus or a herpes virus. In some embodiments, a plurality of rAAV particles is substantially free of one or both of a helper adenovirus or a herpes virus, e.g., a purity of at least about 90%, about 91%, about 92%, about 93%, about 94%, about 95%, about 96%, about 97%, about 98%, about 99%, or more free of one or both of a helper adenovirus or a herpes virus. In some embodiments, a plurality of rAAV particles has a reduced level of adenoviral impurities, e.g., a purity of at least about 50%, about 60%, about 70%, about 80%, about 85%, about 90%, about 91%, about 92%, about 93%, about 94%, about 95%, about 96%, about 97%, about 98%, about 99%, or more free of adenoviral impurities, e.g., relative to rAAV particles produced with a batch complexing method without an in-line complexer as disclosed herein. In some embodiments, a plurality of rAAV particles comprises less than or about 50% adenoviral impurities, e.g., less than or about 45%, 40%, 35%, 30%, 25%, 20%, 15%, 10%, 5%, 4%, 3%, 2%, 1%, or less adenoviral impurities, e.g., relative to rAAV particles produced with a batch complexing method without an in-line complexer as disclosed herein.

In some embodiments, a plurality of rAAV particles produced with methods described herein comprises increased expression of at least one payload, e.g., relative to rAAV particles produced with a batch complexing method without an in-line complexer as disclosed herein. In some embodiments, a plurality of rAAV expresses or comprises at least one payload at a level of about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, or more greater than rAAV particles produced with a batch complexing method without an in-line complexer as disclosed herein.

In some embodiments, a plurality of rAAV particles produced with methods described herein has improved infectivity of a cell or a tissue, e.g., relative to rAAV particles produced with a batch complexing method without an in-line complexer as disclosed herein. In some embodiments, a cell or a tissue infected with a plurality of rAAV particles comprises an increased amount or expression of at least one payload, e.g., an increase of at least about 1%, about 2%, about 3%, about 4%, about 5%, about 10%, about 15%, about 20%, about 25%, about 30%, about 40%, about 45%, about 50%, about 60%, about 70%, about 80%, about 85%, about 90%, about 91%, about 92%, about 93%, about 94%, about 95%, about 96%, about 97%, about 98%, about 99%, or more, e.g., relative to rAAV particles produced with a batch complexing method without an in-line complexer as disclosed herein. In some embodiments, a plurality of rAAV particles produced with methods described herein has improved transfection (e.g., improved transfection efficiency), e.g., relative to rAAV particles produced with a reference helper vector (e.g., an Ad5 vector)). In some embodiments, transfection efficiency is increased by at least about 1%, about 2%, about 3%, about 4%, about 5%, about 10%, about 15%, about 20%, about 25%, about 30%, about 40%, about 45%, about 50%, about 60%, about 70%, about 80%, about 85%, about 90%, about 91%, about 92%, about 93%, about 94%, about 95%, about 96%, about 97%, about 98%, about 99%, or more, e.g., relative to rAAV particles produced with a reference helper vector (e.g., an Ad5 vector, e.g., an Ad5 vector described herein)).

The foregoing methods for producing recombinant vectors are not meant to be limiting, and other suitable methods will be apparent to the skilled artisan.

rAAV Particle Compositions

The present disclosure, among other things, provides a composition comprising a plurality of rAAV particles formed by methods described herein and/or using systems described herein. In some embodiments, a composition comprises a pharmaceutical composition comprising at least one pharmaceutically acceptable component (e.g., a pharmaceutically acceptable carrier, diluent, or excipient). Such pharmaceutical compositions are useful for, among other things, administration to a subject in vivo or ex vivo.

In some embodiments, pharmaceutical compositions also contain a pharmaceutically acceptable carrier, excipient, or diluent. Such excipients include any pharmaceutical agent, e.g., a pharmaceutical agent that does not itself induce an immune response harmful to the individual receiving the composition, and which may be administered without undue toxicity. Pharmaceutically acceptable excipients include, but are not limited to, liquids, such as water, saline, glycerol, sugars, and ethanol. Pharmaceutically acceptable salts can also be included therein, for example, mineral acid salts, such as hydrochlorides, hydrobromides, phosphates, or sulfates; and the salts of organic acids, such as acetates, propionates, malonates, or benzoates. Additionally, auxiliary substances, such as wetting or emulsifying agents or pH buffering substances, may be present in such vehicles.

Pharmaceutical compositions may be provided as a salt and can be formed with many acids, including but not limited to, hydrochloric, sulfuric, acetic, lactic, tartaric, malic, or succinic. Salts tend to be more soluble in aqueous or other protonic solvents than corresponding free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, and isotonic and absorption promoting or delaying agents, compatible with pharmaceutical administration or in vivo contact or delivery. Aqueous and non-aqueous solvents, solutions, and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable, or synthetic oils. Aqueous injection suspensions may contain substances that increase the viscosity of a suspension, such as sodium carboxymethyl cellulose, sorbitol, or dextran. Additionally, suspensions may be prepared as appropriate oil injection suspensions. Suitable lipophilic solvents or vehicles include fatty oils, such as sesame oil, or synthetic fatty acid esters, such as ethyl oleate or triglycerides, or liposomes. Optionally, the suspension may also contain suitable stabilizers or agents that increase solubility to allow for preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

### Administration

The present disclosure, among other things, provides methods of administering a composition (e.g., a pharmaceutical composition) comprising a plurality of rAAV particles formed by methods described herein and/or produced using systems described herein. Compositions (e.g., pharmaceutical compositions) comprising rAAVs produced with the methods described herein or using systems described herein can be used to treat any disease or disorder, e.g., subjects suffering from or susceptible to a disease or disorder described herein. The route and/or mode of administration can vary depending upon the desired results. One with skill in the art (e.g., a physician), is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. Mode of administration is left to discretion of a practitioner.

For example, a composition may be administered by retinal, subretinal, intravitreal, or suprachoroidal injection or infusion. Additional exemplary routes of administration may include, but are not limited to, bronchial (e.g., bronchial instillation), buccal, enteral, interdermal, intra-arterial, intracisterna magna (ICM), intradermal, intragastric, intramedullary, intramuscular, intranasal, intra-parenchymal (e.g., intra-thalamic), intraperitoneal, intrathecal, intravenous, intraventricular, mucosal, nasal, oral, rectal, intraspinal, spinal sub-pial, subcutaneous, sublingual, topical, tracheal (e.g., intratracheal instillation), transdermal, vaginal, and vitreal administration.

Methods and uses disclosed herein include delivery and administration systemically, regionally or locally, or by any route, for example, by injection or infusion. A composition (e.g., a pharmaceutical composition) comprising a plurality of rAAV particles formed by methods described herein may be administered by injection or infusion by any route. For example, a composition may be administered by retinal, subretinal, intravitreal, suprachoroidal, intraspinal, intracisterna magna, or intrathecal injection or infusion.

Delivery of a pharmaceutical composition in vivo may generally be accomplished via injection using a conventional syringe, although other delivery methods such as convection-enhanced delivery can also be used. For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in treatment of patients with certain diseases or disorders may determine the optimal route for administration of vectors described herein.

The disclosure provides methods for introducing a composition (e.g., a pharmaceutical composition) comprising rAAV particles described herein into a cell, a tissue, or an animal. In some embodiments, such methods comprise contacting a cell, a tissue, or an animal with a composition comprising rAAV particles described herein, such that at least one payload is expressed or present in the cell, tissue, or animal.

The disclosure also provides methods for administering a composition (e.g., a pharmaceutical composition) comprising rAAV particles described herein to a subject. In some embodiments, such methods include administering to a subject (e.g., a mammal), a composition comprising rAAV particles described herein, such that at least one payload is expressed or present in the subject (e.g., in a cell or tissue of a subject). In some embodiments, a method includes providing cells of a subject (e.g., a mammal) with a composition (e.g., a pharmaceutical composition) comprising rAAV particles described herein, such that at least one payload is expressed or present in the subject.

A composition (e.g., a pharmaceutical composition) comprising rAAV particles described herein can be administered in a sufficient or effective amount to a subject in need thereof. Doses can vary and depend upon a type, onset, progression, severity, frequency, duration, or probability of disease to which treatment is directed, the clinical endpoint desired, previous or simultaneous treatments, the general health, age, gender, race or immunological competency of the subject, and other factors that will be appreciated by a skilled artisan. Dose amount, number, frequency, or duration may be proportionally increased or reduced, as indicated by any adverse side effects, complications, or other risk factors of treatment and status of the subject. A skilled artisan will appreciate the factors that may influence the dosage and timing required to provide an amount sufficient for providing a therapeutic or prophylactic benefit.

A dose to achieve a therapeutic effect will vary based on several factors including, but not limited to: route of administration, level of payload or payload expression required to achieve a therapeutic effect, specific disease treated, any host immune response, and stability of payload or payload expression. One skilled in the art can determine a dose range to treat a patient having a particular disease or disorder based on the aforementioned factors, as well as other factors.

An effective amount or a sufficient amount can (but need not) be provided in a single administration, may require multiple administrations, and, can (but need not) be, administered alone or in combination with another composition. For example, an amount may be proportionally increased as indicated by need of a subject, type, status, and severity of disease treated or side effects (if any) of treatment. Amounts considered effective also include amounts that result in a reduction of use of another treatment, therapeutic regimen, or protocol.

Accordingly, pharmaceutical compositions include compositions comprising rAAV particles in an effective amount to achieve an intended therapeutic purpose. Determining a therapeutically effective dose is well within the capability of a skilled medical practitioner using techniques and guidance provided herein. Therapeutic doses can depend on, among other factors, age and general condition of a subject, severity of a disease or disorder, and payload amount or expression in a subject. Thus, a therapeutically effective amount in humans will fall in a relatively broad range that may be determined by a medical practitioner based on response of an individual patient to rAAV-based treatment. Pharmaceutical compositions may be delivered to a subject so as to allow production of a payload described herein in vivo by gene- and or cell-based therapies or by ex vivo modification of a patient's or donor's cells.

In some embodiments, a composition (e.g., a pharmaceutical composition) comprising rAAV particles described herein may be administered to a subject once daily, weekly, every 2, 3, or 4 weeks, or even at longer intervals. In some embodiments, a composition (e.g., a pharmaceutical composition) comprising rAAV particles described herein may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments, a composition (e.g., a pharmaceutical composition) comprising rAAV particles described herein may be administered (i) one or more times during an initial time period of up to 2, 4, or 6 weeks or less; followed by (ii) a period of no administration of, e.g., 1, 2, 3, 4, 5, 6, 7, 8, 9, or 10 years. In some embodiments, a subject is monitored before and/or following treatment with a composition (e.g., a pharmaceutical composition) comprising rAAV particles described herein.

### Uses

The present disclosure, among other things, provides methods of delivering a composition (e.g., a pharmaceutical composition) comprising a plurality of rAAV particles as described herein to a cell or tissue. The present disclosure also provides methods of treating a subject with a composition (e.g., a pharmaceutical composition) comprising a plurality of rAAV particles produced using a method or system described herein.

In some embodiments, methods and kits of the present invention may be used for the evaluation and/or monitoring of gene therapy. In some embodiments, gene therapy comprises administration of a composition (e.g., a pharmaceutical composition) comprising a plurality of rAAV particles described herein. In some embodiments, samples for evaluating and/or monitoring gene therapy may be obtained prior to the initiation of gene therapy. In some embodiments, samples are obtained after a first gene therapy treatment or dose. In some embodiments, samples are obtained after the conclusion of gene therapy. In some embodiments, samples are obtained at specific time points, intervals, or any other metric of time before, during, or after gene therapy is performed.

In some embodiments, a composition (e.g., a pharmaceutical composition) comprising a plurality of rAAV particles as described herein is administered to a subject suffering from or at risk of a disease, disorder, or condition. In some embodiments, a composition (e.g., a pharmaceutical composition) comprising a plurality of rAAV particles as described herein is administered in combination with one or more additional therapeutics agents to a subject. In some embodiments, a composition (e.g., a pharmaceutical composition) comprising a plurality of rAAV particles as described herein is contacted with an organ, tissue, or cells ex vivo. The organ, tissue, or cells can be introduced into a subject and can be protected from damage that would otherwise be caused by the recipient's immune system.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### EXEMPLARY EMBODIMENTS

Embodiment 1. A method for transfecting host cells with nucleic acids, the method comprising:
combining nucleic acids with a transfection reagent in an in-line complexer to form complexes that comprise the nucleic acids and the transfection reagent; and
introducing the complexes into a vessel that comprises host cells under conditions that lead to transfection of the host cells with the nucleic acids,
wherein the in-line complexer comprises (a) a first input tubing in communication at a proximal end to a source that comprises the nucleic acids, (b) a second input tubing in communication at a proximal end to a source that comprises the transfection reagent, and (c) an output tubing that is in communication: (i) at a proximal end to a distal end of the first input tubing and a distal end of the second input tubing; and (ii) at a distal end to the vessel that comprises the host cells,
the first input tubing and the second input tubing are each in communication with a pump that has a flow rate of about 1 mL/min to 5000 mL/min, and
the output tubing is about 60 mm to 100,000 mm in length and about 0.3 mm to 250 mm in inner diameter.

Embodiment 2. The method of embodiment 1, wherein the flow rate of the pump is about 10 mL/min to about 500 mL/min.

Embodiment 3. The method of embodiment 1 or 2, wherein the output tubing is about 60 mm to about 10,000 mm in length.

Embodiment 4. The method of any one of embodiment 1-3, wherein the output tubing is about 3 mm to about 26 mm in inner diameter.

Embodiment 5. The method of any one of embodiment 1-4, wherein the output tubing is about 3.2 mm to about 25.4 mm in inner diameter.

Embodiment 6. The method of any one of embodiment 1-4, wherein the output tubing is about 6.35 mm to about 25.4 mm in inner diameter.

Embodiment 7. The method of any one of embodiment 1-4, wherein the output tubing is about 4 mm to about 8 mm in inner diameter.

Embodiment 8. The method of any one of embodiment 1-4, wherein the output tubing is about 20 mm to about 30 mm in inner diameter.

Embodiment 9. The method of anyone of the preceding embodiments, wherein the output tubing is configured in a coil configuration.

Embodiment 10. The method of anyone of the preceding embodiments, wherein the output tubing is at an angle relative to horizontal that allows removal of air bubbles.

Embodiment 11. The method of anyone of the preceding embodiments, wherein the output tubing is at an angle of at least 10 degrees relative to horizontal.

Embodiment 12. The method of any one of the preceding embodiments, wherein a composition comprising the nucleic acids in an amount of about 0.1% to about 10% of the vessel volume is introduced into the first tubing.

Embodiment 13. The method of any one the preceding embodiments, wherein about 100 mL to about 150,000 mL of a composition comprising the nucleic acids is introduced into the first tubing.

Embodiment 14. The method of any one the preceding embodiments, wherein about 150 mL to about 100,000 mL of the composition comprising the nucleic acids is introduced into the first tubing

Embodiment 15. The method of any one of the preceding embodiments, wherein a composition comprising the transfection reagent in an amount of about 0.1% to about 10% of the vessel volume is introduced into the second tubing.

Embodiment 16. The method of any one of the preceding embodiments, wherein about 100 mL to about 150,000 mL of a composition comprising the transfection reagent is introduced into the second tubing.

Embodiment 17. The method of any one of the preceding embodiments, wherein about 150 mL to about 100,000 mL of a composition comprising the transfection reagent is introduced into the second tubing.

Embodiment 18. The method of any one of the preceding embodiments, wherein the composition comprising the nucleic acids and the composition comprising the transfection reagent comprises a combined volume of about 0.1% to about 10% of the vessel volume.

Embodiment 19. The method of any one of the preceding embodiments, wherein the composition comprising the nucleic acids and the composition comprising the transfection reagent comprises a combined volume of about 10% of the vessel volume.

Embodiment 20. The method of any one of the preceding embodiments, wherein the composition comprising the nucleic acids and the composition comprising the transfection reagent comprises a combined volume of about 100 mL to about 1,500,000 mL.

Embodiment 21. The method of any one of the preceding embodiments, wherein the nucleic acids comprise one or more vectors.

Embodiment 22. The method of embodiment 21, wherein the nucleic acids comprise one or more vectors encoding:
(i) at least one payload flanked by an AAV inverted terminal repeat (ITR) on either side of the at least one payload,
(ii) at least one AAV Rep polypeptide,
(iii) at least one AAV Cap polypeptide, and/or
(iv) at least one Adenoviral helper polypeptide.

Embodiment 23. The method of embodiment 21 or 22, wherein the one or more vectors comprise:
(i) a first vector encoding at least one payload flanked by an AAV ITR on either side of the at least one payload,
(ii) a second vector encoding at least one AAV Rep polypeptide and at least one AAV Cap polypeptide, and/or
(iii) a third vector encoding at least one Adenoviral helper polypeptide.

Embodiment 24. The method of any one of embodiments 21-23, wherein the one or more vectors comprise:
(i) a first vector encoding at least one AAV Cap polypeptide and at least one payload flanked by an AAV ITR on either side of the at least one payload; and
(ii) a second vector encoding at least one Adenoviral helper polypeptide and at least one AAV Rep polypeptide.

Embodiment 25. The method of any one of embodiments 21-24, wherein the at least one Adenoviral helper polypeptide comprises one, two, three, or four of E1, E2A, E4orf6, or VA RNA polypeptides.

Embodiment 26. The method of any one of embodiments 21-25, wherein the AAV Cap polypeptide comprises an AAV1, AAV2, AAV3A, AAB3B, AAV4, AAV5, AAV6, AAV7, AAV8, AAV9, AAVrh10, AAV-DJ, AAV-PHP.B, AAV-PHP.N, or AAV-PHP.S Cap polypeptide, or a variant of any of the foregoing.

Embodiment 27. The method of any one of embodiments 21-26, wherein the AAV Rep polypeptide comprises an AAV2 Rep polypeptide, or a variant thereof.

Embodiment 28. The method of any one of embodiments 21-27, wherein the AAV ITR comprises an AAV2 ITR, or a variant thereof.

Embodiment 29. The method of any one of the preceding embodiments, further comprising culturing the host cells under conditions suitable for producing recombinant AAV (rAAV) particles.

Embodiment 30. The method of embodiment 29, further comprising collecting the rAAV particles from the vessel.

Embodiment 31. The method of embodiment 30, wherein the rAAV particles are collected without lysing the host cells.

Embodiment 32. The method of any one of the preceding embodiments, wherein the nucleic acids are diluted in cell culture media.

Embodiment 33. The method of any one of the preceding embodiments, wherein the transfection reagent comprises a polymer, or a lipid, or both.

Embodiment 34. The method of embodiment 33, wherein the transfection reagent comprises polyethyleneimine (PEI), FectoVIR, TransIT-VirusGEN, or a combination thereof.

Embodiment 35. The method of embodiment 33 or 34, wherein the transfection reagent is or comprises PEI.

Embodiment 36. The method of any one of the preceding embodiments, wherein the complexes have an average diameter of about 100 nm to about 1000 nm.

Embodiment 37. The method of any one of the preceding embodiments, wherein the complexes have an average diameter of less than 700 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm.

Embodiment 38. The method of any one of the preceding embodiments, wherein at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to about 1000 nm.

Embodiment 39. The method of any one of the preceding embodiments, wherein it takes between about 30 seconds and about 3600 seconds for the nucleic acids, transfection reagent and/or complexes comprising the nucleic acid and transfection reagent to travel the length of the output tubing.

Embodiment 40. The method of embodiment 39, wherein it takes about 30 seconds to about 3600 seconds, for the nucleic acids, transfection reagent and/or complexes comprising the nucleic acid and transfection reagent to travel the length of the output tubing.

Embodiment 41. The method of embodiment 39 or 40, wherein it takes at least 30 seconds for the nucleic acids, transfection reagent and/or complexes comprising the nucleic acid and transfection reagent to travel the length of the output tubing.

Embodiment 42. The method of embodiment 39 or 40, wherein it takes no more than 3600 seconds for the nucleic acids, transfection reagent and/or complexes comprising the nucleic acid and transfection reagent to travel the length of the output tubing.

Embodiment 43. The method of embodiment 39 or 40 wherein it takes about 150 seconds for the nucleic acids, transfection reagent and/or complexes comprising the nucleic acid and transfection reagent to travel the length of the output tubing.

Embodiment 44. The method of any one of the preceding embodiments wherein the shear rate of a solution flowing through the first input tubing, the second input tubing and/or the output tubing is about 5 s⁻¹ to about 30 s⁻¹.

Embodiment 45. The method of embodiment 44, wherein the shear rate of a solution flowing through the first input tubing, the second input tubing and/or the output tubing is about 10 s⁻¹ to about 20 s⁻¹.

Embodiment 46. The method of any one of the preceding embodiments, wherein the in-line complexer further comprises: a mixer.

Embodiment 47. The method of embodiment 46, wherein the mixer is or comprises a static mixer.

Embodiment 48. The method of embodiment 47, wherein the static mixer comprises a nozzle mixer, an injector, an orifice, a valve, a pump, or a combination thereof.

Embodiment 49. The method of embodiment 47 or 48, wherein the static mixer is or comprise a nozzle mixer.

Embodiment 50. The method of any one of embodiments 46-49, wherein the mixer is in communication with the distal end of the first and second input tubings and the proximal end of the output tubing.

Embodiment 51. The method of any one of embodiments 46-50, wherein the output tubing comprises an upstream and a downstream portion that are located upstream and downstream of the mixer.

Embodiment 52. The method of any one of the preceding embodiments, wherein the in-line complexer further comprises one or more scales.

Embodiment 53. The method of embodiment 52, wherein the in-line complexer comprises a scale attached to a source in communication with the first tubing.

Embodiment 54. The method of embodiment 52 or 53, wherein the in-line complexer comprises a scale attached to a source in communication with the second tubing.

Embodiment 55. The method of any one of the preceding embodiments, wherein the vessel is a bioreactor.

Embodiment 56. The method of embodiment 55, wherein the bioreactor comprises one or both of:
(i) at least 1×10⁵ host cells; or
(ii) at least 1 L of culture media.

Embodiment 57. The method of embodiment 56, wherein the host cells are or comprise viable cells (vc).

Embodiment 58. The method of embodiment 56 or 57, wherein the bioreactor comprises one or both of:
(i) about 1×10⁶ vc/mL to about 5×10⁶ vc/mL; or
(ii) about 3 L to about 10,000 L culture media.

Embodiment 59. The method of any one of embodiments 55-58, wherein the bioreactor is a continuous flow bioreactor, a batch process bioreactor, a perfusion bioreactor, or a fed batch bioreactor.

Embodiment 60. The method of any one of embodiments 55-59, wherein the bioreactor comprises one or more probes.

Embodiment 61. The method of embodiment 60, wherein the probe is a biocapacitance probe.

Embodiment 62. The method of any one of embodiments 55-61, wherein the bioreactor comprises one or more scales.

Embodiment 63 The method of any one of the preceding embodiments, wherein the host cells are suspension adapted host cells.

Embodiment 64. The method of embodiment 63, wherein the host cells are mammalian cells.

Embodiment 65. The method of embodiment 64, wherein the mammalian cells are HEK293 cells, CHO-K, or HeLa cells.

Embodiment 66. A system for transfecting host cells with nucleic acids, the system comprising an in-line complexer and a vessel comprising host cells, wherein:
the in-line complexer comprises (a) a first input tubing in communication at a proximal end to a source that comprises nucleic acids, (b) a second input tubing in communication at a proximal end to a source that comprises a transfection reagent, and (c) an output tubing that is in communication (i) at a proximal end to a distal end of the first input tubing and a distal end of the second input tubing and (ii) at a distal end to the vessel that comprises host cells,
the first input tubing and the second input tubing are each in communication with a pump that has a flow rate of about 1 mL/min to about 5000 mL/min, and
the output tubing is about 60 mm to 100,000 mm in length and about 0.3 mm to 250 mm in inner diameter.

Embodiment 67. The system of embodiment 66, wherein the system is used in a method for transfecting host cells with nucleic acids, said method comprising combining nucleic acids with a transfection reagent to form complexes that comprise the nucleic acids and the transfection reagent.

Embodiment 68. The system of embodiment 66 or 679, wherein the complexes have an average diameter of about 100 nm to about 1000 nm.

Embodiment 69. The system of any one of embodiments 66-68, wherein at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to 1000 nm.

Embodiment 70. The system of any one of embodiments 66-69, wherein it takes between about 30 seconds and about 600 seconds for the nucleic acids, transfection reagent and/or complexes comprising the nucleic acid and transfection reagent to travel the length of the output tubing.

Embodiment 71. The system of any one of embodiments 66-70, wherein it takes between about 30 seconds and about 600 seconds for the nucleic acids, transfection reagent and/or complexes comprising the nucleic acid and transfection reagent to travel the length of the output tubing.

Embodiment 72. The system of embodiment 71, wherein it takes at least 30 seconds for the nucleic acids, transfection reagent and/or complexes comprising the nucleic acid and transfection reagent to travel the length of the output tubing.

Embodiment 73. The system of embodiment 71, wherein it takes no more than 600 seconds for the nucleic acids, transfection reagent and/or complexes comprising the nucleic acid and transfection reagent to travel the length of the output tubing.

Embodiment 74. The system of any one of embodiments 70-73, wherein it takes about 150 seconds for the nucleic acids, transfection reagent and/or complexes comprising the nucleic acid and transfection reagent to travel the length of the output tubing.

Embodiment 75. The system of any one of embodiments 66-74, wherein the shear rate of a solution flowing through the first input tubing, the second input tubing and/or the output tubing is about 5 s⁻¹ to about 30 s⁻¹.

Embodiment 76. The method of embodiment 75, wherein the shear rate of a solution flowing through the first input tubing, the second input tubing and/or the output tubing is about 10 s⁻¹ to about 20 s⁻¹.

Embodiment 77. The system of any one of embodiments 48-54, wherein the in-line complexer further comprises a mixer.

Embodiment 78. The system of embodiment 77, wherein the mixer is or comprises a static mixer.

Embodiment 79. The system of embodiment 78, wherein the static mixer comprises a nozzle mixer, an injector, an orifice, a valve, a pump, or a combination thereof.

Embodiment 80. The system of embodiment 78 or 79, wherein the static mixer is or comprise a nozzle mixer.

Embodiment 81. The system of any one of embodiments 77-80, wherein the mixer is in communication with the distal end of the first and second input tubings and the proximal end of the output tubing.

Embodiment 82. The system of embodiment 81, wherein the output tubing comprises an upstream and a downstream portion that are located upstream and downstream of the mixer.

Embodiment 83. The system of any one of embodiments 66-81, wherein the in-line complexer further comprises one or more scales.

Embodiment 84. The system of embodiment 83, wherein the in-line complexer comprises a scale attached to a source in communication with the first tubing.

Embodiment 85. The system of embodiment 83, wherein the in-line complexer comprises a scale attached to a source in communication with the second tubing.

Embodiment 86. The system of any one of embodiments 66-85, wherein the vessel is a bioreactor.

Embodiment 87. The system of embodiment 86, wherein the bioreactor comprises one or both of.
(i) at least 1×10⁵ host cells; or
(ii) at least 1 L of culture media.

Embodiment 88. The system of embodiment 87, wherein the host cells are or comprise viable cells (vc).

Embodiment 89. The system of any one of embodiments 86-88, wherein the bioreactor comprises one or both of:
(i) about 1×10⁶ vc/mL to about 5×10⁶ vc/mL; or
(ii) about 3 L to about 10,000 L culture media.

Embodiment 90. The system of any one of embodiments 86-89, wherein the bioreactor is selected from a continuous flow bioreactor, a batch process bioreactor, a perfusion bioreactor, or a fed batch bioreactor.

Embodiment 91. The system of any one of embodiments 66-90, wherein the bioreactor comprises one or more probes

Embodiment 92. The system of embodiment 91, wherein the probe is a biocapacitance probe.

Embodiment 93. The system of any one of embodiments 66-92, wherein the bioreactor comprises one or more scales.

Embodiment 94. A transfection complex produced by the method of any one of embodiments 1-65, or the system of any one of embodiments 66-93.

Embodiment 95. A culture comprising a plurality of host cells and the transfection complex of embodiment 94.

Embodiment 96. A bioreactor comprising the culture of embodiment 95.

Embodiment 97. The bioreactor of embodiment 96, wherein the bioreactor comprises one or both of:
(i) at least 1×10⁵ host cells; or
(ii) at least 1 L of culture media.

Embodiment 98. The bioreactor of embodiment 97, wherein the host cells are or comprise viable cells (vc).

Embodiment 99. The bioreactor of embodiment 97 or 98, wherein the bioreactor comprises one or both of:
(i) about 1×10⁶ vc/mL to about 5×10⁶ vc/mL; or
(ii) about 3 L to about 10,000 L culture media.

Embodiment 100. The bioreactor of any one of embodiments 96-99, wherein the bioreactor is selected from a continuous flow bioreactor, a batch process bioreactor, a perfusion bioreactor, and a fed batch bioreactor

Embodiment 101. The bioreactor of any one of embodiments 96-100, wherein the bioreactor is held under conditions suitable for formation of a plurality of rAAV particles.

Embodiment 102. The bioreactor of any one of embodiments 96-100, wherein the bioreactor comprises one or more scales.

Embodiment 103. The bioreactor of any one of embodiments 96-100, wherein the bioreactor comprises one or more probes.

Embodiment 104. The bioreactor of embodiment 103, wherein the probe is a biocapacitance probe.

Embodiment 105. A composition comprising a plurality of rAAV particles produced by the method of any one of embodiments 1-65, or using a system of any one of embodiments 66-93.

Embodiment 106. A pharmaceutical composition comprising the composition of embodiment 105 and a pharmaceutically acceptable component.

Embodiment 107. A method of administering the pharmaceutical composition of embodiment 106 to a subject.

Embodiment 108. The method of embodiment 107, wherein the subject is a mammal.

Embodiment 109. The method of embodiment 107, wherein the subject is a human.

### EXAMPLES

The following example is put forth so as to provide those of ordinary skill in the art with a complete disclosure and description of how to make and use the present disclosure, and is not intended to limit the scope of what the inventors regard as their discovery nor are they intended to represent that the experiments below are all or the only experiments performed.

### Example 1: Host Cell Transfection with In-Line Complexation

### Introduction

In the suspension transient transfection (sTT) process for AAV gene therapy production, the transfection step remains the most complicated and challenging step in the process and the largest source of process variability. In the exemplary sTT process described herein, transfection describes the process of using a commercially available polymer, e.g., a PEI polymer such as PEIpro (Polyplus), to deliver plasmid DNA across the cell membrane for gene expression. When the polymer encounters the DNA, the opposite charges of the DNA and the PEI result in their binding to form nanoparticles which mediate the movement of the DNA into the cell. The DNA and PEI are both diluted in an optimized carrier media that facilitates proper nanoparticle formation when both of the diluted solutions are mixed.

The primary challenge with the formation of these nanoparticles is their time stability, as peak productivity is seen at about a 5-minute incubation time after the PEI and DNA solutions meet and a decline in productivity is observed with increasing time. This presents a considerable pain point in large-scale manufacturing operations, where the volumes for the DNA and PEI solutions get much larger and completing the entire operation within 5 minutes poses a technical problem. While at the 3 liter scale for process development, the entire nanoparticle addition to the bioreactor can be completed in 30 seconds or less, at the 1000 liter scale for a potential manufacturing scenario, the addition could take up to 15 minutes, which would result in a reduction in productivity and a loss of process robustness.

The novel improvement of the in-line complexing method disclosed herein is the elimination of this hold time for complexing, which de-bottlenecks the transfection step and provides more process consistency. By introducing the diluted PEI and DNA solutions through pumps and a static mixer, the hold time can be controlled in a reaction coil for a precise complex time prior to addition to the bioreactor, no matter the addition volume or scale. The in-line complexation method also allows for better process consistency, as operators could accurately ensure a pre-determined, e.g., a 5-minute, complex time in every single production run through defined set points for the PEI and DNA pumps. While using a batch complexing method where the nanoparticles are formed all at once and are added to the bioreactor all at once, it is difficult to maintain the same complex and addition times between batches even using the same operators and equipment, which highlights a key challenge to maintaining process consistency during a tech transfer for manufacturing. The in-line complex method disclosed herein addresses this challenge by providing a consistent and scalable alternative to the traditional batch strategy, and could enable optimal PEI/DNA nanoparticles at any scale bioreactor without the need for a time critical operation.

### Methods:

For the 3000 ml (3 liter) bioreactor model controlling at a DNA/PEI incubation time of 5 minutes, a silicone tubing of 6.4 mm inner diameter (ID) and a length of 3.77 m was used for the complexing step between the joining of the 2 solutions and the bioreactor. A 150 mL solution of Opti-MEM media (Gibco) with the addition of the triple plasmids (Rep/Cap, AAV-gene of interest, and Ad2 helper) used for transfection was placed in a sterile PETG bottle with a 3.18 mm ID tubing diptube. Another mL150 mL solution of OptiMEM was placed in a separate PETG bottle with the aliquot of PEIpro (Polyplus). Both outlet tubings from the OptiMEM solution bottles were placed in separate peristaltic pumps and on the outlet side of the pumps, the tubing met with a “Y” shaped tubing connector, the outlet of which was the 3.77 m, 6.4 mm ID tubing.

To initiate the transfection, both peristaltic pumps were turned on at a target flow rate of 12.6 mL/min, which resulted in a flow rate of 25.2 mL/min through the 6.4 mm ID tubing and a 5 min residence time prior to addition to the bioreactor. This flow rate was shown to produce nanoparticles of consistent size to the traditional batch complexing method. The peristaltic pump ran for a total of 17 minutes for a total addition time to the bioreactor of around 12 minutes. This 300 mL transfection addition to the 3000 mL bioreactor was a 10% total addition.

While scaling up this approach to larger bioreactor with greater transfection complex volumes, wider tubing sizes will be required for the total addition time to stay similar to the 3 liter bioreactor and traditional batch complexing transfection methods. In some embodiments, with the larger tubing diameters, it is expected that the scaling factor to be used will be shear rate which is calculated based on the following equation:

$$Shear rate ( γ ) = \frac{8 * linear fluid velovity ( v )}{tubing inner diameter ( d )}$$

Scaling the tubing flow rates, lengths and diameters while maintaining equivalent shear rates is expected to allow for similar hydrodynamic conditions for the nanoparticle formation and as a result consistent complex formation to enable robust cell culture transfection.

### Results:

An experiment was performed using the in-line complex method described above to understand how consistent the DNA/PEI nanoparticles formed are with respect to time after starting the pumps and to understand how the nanoparticles compared to traditional batch complexing methods. A Dynamic Light Scattering instrument (Zetasizer Ultra, Malvern Panalytical) was used to measure the size of the DNA/PEI complex at various time point with both methods.

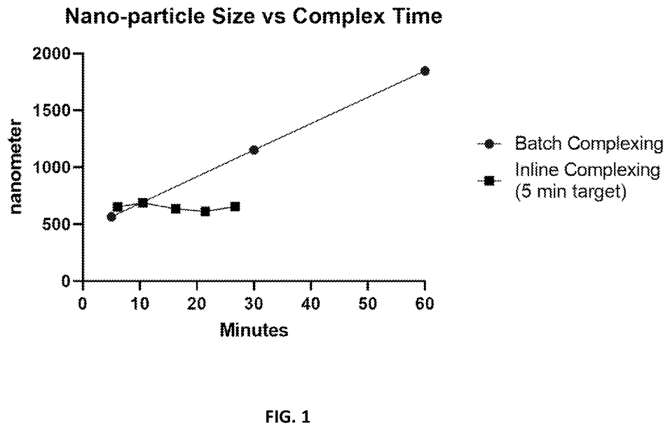

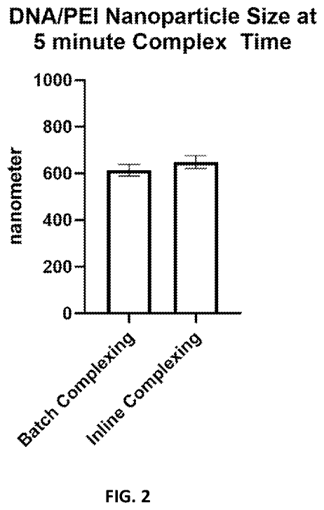

FIG. 1 shows the stability of the nanoparticles with respect to time, showing that at approximately 30 minutes, the in-line complexer delivered consistently sized nanoparticles, while the complexes formed in a single batch have aggregated and roughly doubled in size. This demonstrates the effectiveness of the in-line complexer for robustly delivering large volumes of DNA/PEI complex over a longer period of time compared to batch complexing where the total addition of complex to the bioreactor is the rate limiting step. FIG. 2 compares the sizes of the nanoparticle complex formed between both methods at a 5-minute incubation time. In-line complexing produces nanoparticles slightly larger than those produced through the batch method, but are very similar with respect to the rate of aggregation of these nanoparticles, which shows the comparability between the in-line complex and the batch complex.

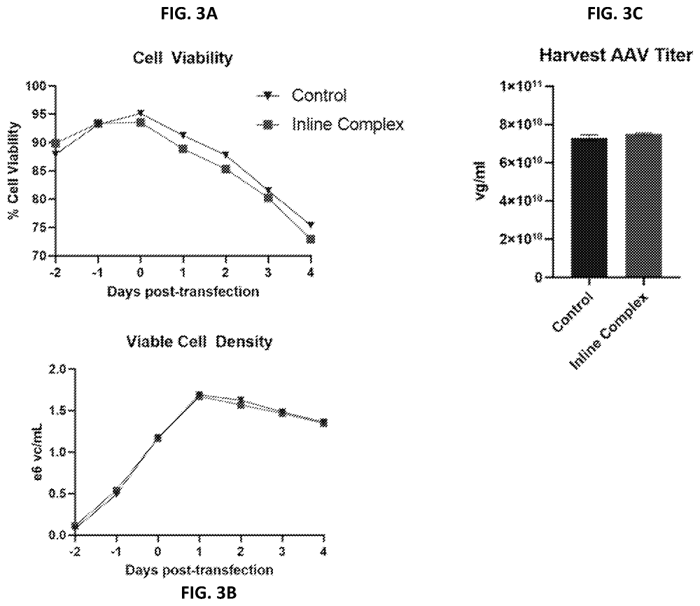

A further experiment was performed to test the performance of these DNA/PEI complexes in the 3 liter bioreactor compared to the batch complexing method. Identical cell cultures, control parameters, media, plasmid and PEIpro lots were used for the comparison. The control vessel utilized the batch complexing method in which the DNA and PEI solutions were mixed together and held for a 5-minute hold step prior to addition to the bioreactor. The addition of the 300 mL batch complex volume to the 3000 mL (3 liter) bioreactor typically takes around 30 seconds to complete. The in-line complex condition used the methods described above, with a target incubation time of 5 minutes. Comparable cell culture performance and harvest titer were observed with both methods (FIGS. 3A-3C). This data demonstrates the equivalency of both methods for complexing of the DNA and PEI and subsequent delivery of the plasmid DNA into the cell for viral production. Based on the equivalency of both methods, the in-line complex method could be used in place of the batch complex method to achieve robust transfections at much larger production volumes.

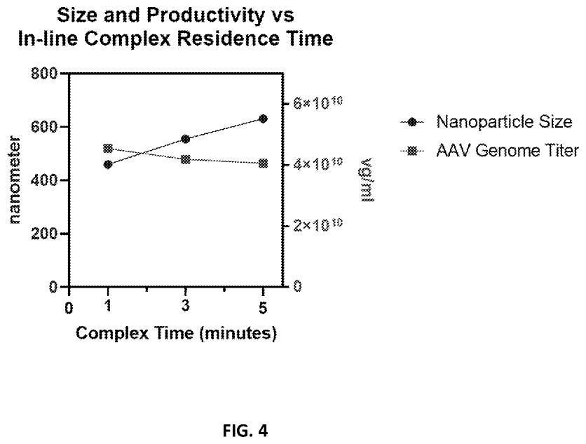

To evaluate the potential for reduced complexation time in the in-line complex, which could provide the advantage of reduced tubing lengths and addition times at larger bioreactor volumes, the following experiment was performed. The in-line complex method described in this experiment was used at the same flow rates, except nanoparticle complexes were removed at different tubing lengths equivalent to 1-minute, 3-minute and 5-minute complex times. These time course samples were used to transfect two (2) replicate shake flask cultures for each of the three (3) time points as well as take DLS readings, which are shown in FIG. 4. The data shows that decreased complexing time in the in-line complexer produces smaller nanoparticles via less aggregation time and positively impacts viral production. This data supports the scaling up of the in-line complexer device to, e.g., 1000 liter and 2000 liter bioreactors, because it could enable shorter tubing lengths and reduced complexation times.

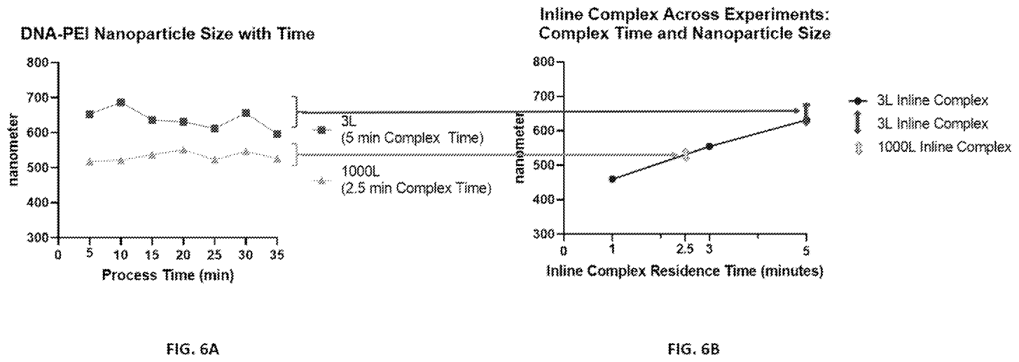

FIGS. 6A-6B show the size of the DNA-PEI nanoparticles formed over time with in-line complexation using a 3 L bioreactor or a 1000 L bioreactor. The data shows that the size of nanoparticles are stable across the duration of the run with both bioreactors (FIG. 6A). In addition, comparing the data in FIG. 6A with the data in FIG. 6B shows that the nanoparticle sizes formed with the 3 L bioreactor and the 1000 L bioreactor are consistent when a similar complex time is used. A complex time of 2.5 minutes in a 1000 L bioreactor produced nanoparticles of about 500 nm (see 1000 L data in FIG. 6A) which is similar to the size of nanoparticles produced with a 3 L bioreactor with a complex time of 2.5 minutes (as shown in FIG. 6B).

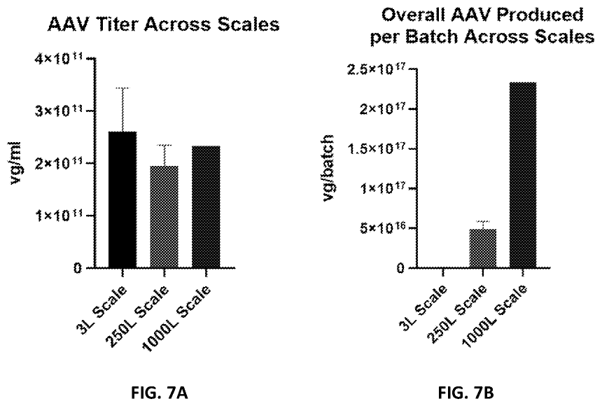

FIG. 7A shows that the viral titer produced from transfection with in-line complexation is similar across bioreactors of various sizes—from 3 L to 1000 L. FIG. 7B shows that the amount of AAV particles produced with in line complexation correlates with the size of bioreactor with the 1000 L bioreactor producing the highest amount of AAV particles.

This data demonstrates the scalability of the in-line complexation process.

### Discussion:

Based on the data shown herein, the disclosed in-line complexer provides an alternative to batch DNA and PEI complexing that is scalable to much larger volumes while maintaining robustness between production runs. The main challenge to batch complexing is the time stability of the nanoparticle complexes, because the longer the DNA and PEI remain together, the more the complexes will aggregate and impact the efficiency of the transfection step and ultimately the viral productivity. By complexing the DNA and PEI using an in-line mixing device, a precise hold time can be achieved through the length of the device, and the DNA and PEI's residence time can be controlled. This will allow for a transfection step that may take longer, but will maintain a more controlled addition of the complex into the bioreactor. In proof-of-concept studies at the 3 liter scale, the complexes generated through in-line complexing were shown to be equivalent to those generated through the standard batch method, demonstrating the technology's potential to be implemented at larger scales. Experiments to reduce the incubation time for the PEI and the DNA showed favorable results in the in-line complexer, particularly at the 1-minute incubation time, which is unrealistic using batch complexing at any scale larger than 3 liter. In batch complexing for a 2000 liter bioreactor, a flow rate of 200 liters per minute would have to be achieved in order to replicate this same addition, which is not feasible for a manufacturing process.

Aside from the scalability advantage with in-line complexing, this method would remove much of the manual operations from the process in favor of an automated and standardized operation. Having the conditions of the nanoparticle formation controlled in the tubing at a defined flow and shear rate allows for not only consistency within a single production from start to finish but also consistency between different productions as long as the conditions in the complexing step are maintained. This robustness will play a key role in standardizing the sTT process for consistent performance in a manufacturing scenario.

We claim:

1. A method for transfecting host cells with nucleic acids, the method comprising:
combining nucleic acids with a transfection reagent in an in-line complexer to form complexes that comprise the nucleic acids and the transfection reagent; and
introducing the complexes into a vessel that comprises host cells under conditions that lead to transfection of the host cells with the nucleic acids,
wherein
the in-line complexer comprises (a) a first input tubing in communication at a proximal end to a source that comprises the nucleic acids, (b) a second input tubing in communication at a proximal end to a source that comprises the transfection reagent, and (c) an output tubing that is in communication: (i) at a proximal end to a distal end of the first input tubing and a distal end of the second input tubing; and (ii) at a distal end to the vessel that comprises the host cells,
the first input tubing and the second input tubing are each in communication with a pump that has a flow rate of about 1 mL/min to 5000 mL/min, and
the output tubing is about 60 mm to 100,000 mm in length and about 0.3 mm to 250 mm in inner diameter.

2. The method of claim 1, wherein the output tubing is configured in a coil configuration.

3. The method of claim 1 or 2, wherein the output tubing is at an angle of at least 10 degrees relative to horizontal.

4. The method of any one of claims 1-3, wherein:
(i) a composition comprising the nucleic acids in an amount of about 0.1% to about 10% of the vessel volume is introduced into the first tubing; and/or
(ii) about 100 mL to about 150,000 mL of a composition comprising the nucleic acids is introduced into the first tubing.

5. The method of any one of claims 1-4, wherein:
(i) a composition comprising the transfection reagent in an amount of about 0.1% to about 10% of the vessel volume is introduced into the second tubing; and/or
(ii) about 100 mL to about 150,000 mL of a composition comprising the transfection reagent is introduced into the second tubing.

6. The method of any one of claims 1-5 wherein:
(i) the composition comprising the nucleic acids and the composition comprising the transfection reagent comprises a combined volume of about 0.1% to about 10% of the vessel volume; and/or
(ii) the composition comprising the nucleic acids and the composition comprising the transfection reagent comprises a combined volume of about 100 mL to about 1,500,000 mL.

7. The method of any one of the preceding claims, wherein the nucleic acids comprise one or more vectors.

8. The method of claim 7, wherein the nucleic acids comprise one or more vectors encoding:
(i) at least one payload flanked by an AAV inverted terminal repeat (ITR) on either side of the at least one payload,
(ii) at least one AAV Rep polypeptide,
(iii) at least one AAV Cap polypeptide, and/or
(iv) at least one Adenoviral helper polypeptide.

9. The method of claim 7 or 8, wherein the one or more vectors comprise:
(i) a first vector encoding at least one payload flanked by an AAV ITR on either side of the at least one payload,
(ii) a second vector encoding at least one AAV Rep polypeptide and at least one AAV Cap polypeptide, and/or
(iii) a third vector encoding at least one Adenoviral helper polypeptide.

10. The method of any one of claims 7-9, wherein the one or more vectors comprise:
(i) a first vector encoding at least one AAV Cap polypeptide and at least one payload flanked by an AAV ITR on either side of the at least one payload; and
(ii) a second vector encoding at least one Adenoviral helper polypeptide and at least one AAV Rep polypeptide.

11. The method of any one of claims 7-10, wherein the AAV ITR comprises an AAV2 ITR, or a variant thereof.

12. The method of any one of the preceding claims, further comprising culturing the host cells under conditions suitable for producing recombinant AAV (rAAV) particles.

13. The method of claim 12, further comprising collecting the rAAV particles from the vessel.

14. The method of claim 3, wherein the rAAV particles are collected without lysing the host cells.

15. The method of any one of the preceding claims, wherein the nucleic acids are diluted in cell culture media.

16. The method of any one of the preceding claims, wherein the transfection reagent comprises a polymer, or a lipid, or both.

17. The method of claim 16, wherein the transfection reagent comprises polyethyleneimine (PEI), FectoVIR, TransIT-VirusGEN, or a combination thereof.

18. The method of claim 16 or 17, wherein the transfection reagent is or comprises PEI.

19. The method of any one of the preceding claims, wherein the complexes have an average diameter of about 100 nm to about 1000 nm.

20. The method of any one of claims 1-19, wherein the complexes have an average diameter of less than 700 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm.

21. The method of any one of claims 1-20, wherein at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to about 1000 nm.

22. The method of any one of the preceding claims, wherein it takes between about 30 seconds and about 3600 seconds for the nucleic acids, transfection reagent and/or complexes comprising the nucleic acid and transfection reagent to travel the length of the output tubing.

23. The method of claim 21 or 22, wherein it takes about 150 seconds for the nucleic acids, transfection reagent and/or complexes comprising the nucleic acid and transfection reagent to travel the length of the output tubing.

24. The method of any one of the preceding claims, wherein the shear rate of a solution flowing through the first input tubing, the second input tubing and/or the output tubing is about 5 s⁻¹ to about 30 s⁻¹.

25. The method of claim 24, wherein the shear rate of a solution flowing through the first input tubing, the second input tubing and/or the output tubing is about 10 s⁻¹ to about 20 s⁻¹.

26. The method of any one of the preceding claims, wherein the in-line complexer further comprises: (a) a mixer; and/or (b) one or more scales.

27. The method of claim 26, wherein the mixer is or comprises a static mixer.

28. The method of any one of the preceding claims, wherein the vessel is a bioreactor.

29. The method of claim 28 wherein the bioreactor comprises one or both of:
(i) at least 1×10⁵ host cells; or
(ii) at least 1 L of culture media.

30. The method of claim 29, wherein the host cells are or comprise viable cells (vc).

31. The method of claim 29 or 30, wherein the bioreactor comprises one or both of:
(i) about 1×10⁶ vc/mL to about 5×10⁶ vc/mL; or
(ii) about 3 L to about 10,000 L culture media.

32. The method of any one of claims 29-31, wherein the bioreactor is a continuous flow bioreactor, a batch process bioreactor, a perfusion bioreactor, or a fed batch bioreactor.

33. The method of any one of claims 29-32, wherein the bioreactor comprises one or more probes and/or one or more scales.

34. The method of any one of the preceding claims, wherein the host cells are suspension adapted host cells.

35. The method of claim 34, wherein the host cells are mammalian cells.

36. The method of claim 35, wherein the mammalian cells are HEK293 cells, CHO-K, or HeLa cells.

37. A system for transfecting host cells with nucleic acids, the system comprising an in-line complexer and a vessel comprising host cells, wherein:
the in-line complexer comprises (a) a first input tubing in communication at a proximal end to a source that comprises nucleic acids, (b) a second input tubing in communication at a proximal end to a source that comprises a transfection reagent, and (c) an output tubing that is in communication (i) at a proximal end to a distal end of the first input tubing and a distal end of the second input tubing and (ii) at a distal end to the vessel that comprises host cells,
the first input tubing and the second input tubing are each in communication with a pump that has a flow rate of about 1 mL/min to about 5000 mL/min, and
the output tubing is about 60 mm to 100,000 mm in length and about 0.3 mm to 250 mm in inner diameter.

38. The system of claim 37, wherein the system is used in a method for transfecting host cells with nucleic acids, said method comprising combining nucleic acids with a transfection reagent to form complexes that comprise the nucleic acids and the transfection reagent.

39. The system of claim 37 or 38, wherein:
(a) the complexes have an average diameter of about 100 nm to about 1000 nm; and/or
(b) at least 10%, at least 20%, at least 30%, at least 40%, at least 50%, at least 60%, at least 70%, at least 80%, at least 90%, at least 95%, at least 96%, at least 97%, at least 98% or at least 99% of the complexes have a diameter of about 100 nm to 1000 nm.

40. The system of any one of claims 37-39, wherein it takes between about 30 seconds and about 600 seconds for the nucleic acids, transfection reagent and/or complexes comprising the nucleic acid and transfection reagent to travel the length of the output tubing.

41. The system of any one of claims 37-40, wherein the shear rate of a solution flowing through the first input tubing, the second input tubing and/or the output tubing is about 5 s⁻¹ to about 30 s⁻¹.

42. The system of any one of claims 37-41, wherein the in-line complexer further comprises: (a) a mixer; and/or (b) one or more scales.

43. The system of any one of claims 37-42, wherein the vessel is a bioreactor.

44. The system of claim 43, wherein the bioreactor comprises one or both of:
(i) at least 1×10⁵ host cells; or
(ii) at least 1 L of culture media.

45. The system of claim 44, wherein the host cells are or comprise viable cells (vc).

46. The system of any one of claims 43-45, wherein the bioreactor is selected from a continuous flow bioreactor, a batch process bioreactor, a perfusion bioreactor, or a fed batch bioreactor.

47. A transfection complex produced by the method of any one of claims 1-36, or the system of any one of claims 37-46.

48. A culture comprising a plurality of host cells and the transfection complex of claim 47.

49. A bioreactor comprising the culture of claim 48.

50. A composition comprising a plurality of rAAV particles produced by the method of any one of claims 1-36, or using a system of any one of claims 37-46.

51. A pharmaceutical composition comprising the composition of claim 50 and a pharmaceutically acceptable component.

52. A method of administering the pharmaceutical composition of claim 51 to a subject.

53. The method of claim 52, wherein the subject is a mammal.
